(19) United States
(12) Patent Application Publication
(10) Pub. No.: US 2004/0034076 A1
(43) Pub. Date: Feb. 19, 2004

(54) Novel carbamates and carbamides, production and use thereof as endothelin receptor antagonists

(76) Inventors: Wilhelm Amberg, Mannheim-Seckenheim (DE); Georg Kettschau, Mannheim (DE)

Correspondence Address: Keil & Weinkauf, 1101 Connecticut Avenue N W, Suite 620, Washington, DC, 20036, US

(21) Appl. No.: 10/296,443

(22) PCT Filed: May 18, 2001
(86) PCT No.: PCT/EP01/05742

(30) Foreign Application Priority Data
May 25, 2000 (DE) 10025728.3

(51) Int. Cl. A61K 31/4196; A61K 31/4162; A61K 31/4172

(52) U.S. Cl. 514/381; 514/383; 514/400; 514/406; 514/408; 548/254; 548/265.2; 548/335.5; 548/370.1; 548/557

(57) ABSTRACT

The present invention relates to carbamates and urea derivatives of the formula I

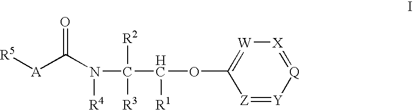

where the substituents have the meanings explained in the description, and to their preparation and use as endothelin receptor antagonists.

The present invention relates to novel carbamate and urea derivatives, their preparation and use as endothelin receptor antagonists.

Endothelih receptor antagonists of the 3,3-disubstituted propionic acids structural type are described in numerous patent applications (WO 95/ 26716, WO 96/11914, WO 97/12878, WO 97/38980, WO 97/38981, WO 97/38982, WO 98/09953, WO 99/23078 and WO 99/42453). Other representatives of the 3,3-disubstituted propionic acid derivatives exhibit a herbicidal action and are therefore of interest as crop protection agents (WO 94/25442, WO 96/00219, DE 4035758, EP 0481512).

WO 98/58916 describes endothelin receptor antagonists of the 3,3-disubstituted propionic acids type, in which C-3 is additionally functionalized by a nitrogen-containing group (such as azido or amino).

It is an object of the present invention to make available endothelin receptor antagonists which bind to the ET_{A} and/or the ET_{B} receptor subtypes. It was surprisingly found here that compounds in which the abovementioned nitrogen is on the C-3 constituent of a carbamate or urea radical have advantageous pharmacological properties.

The present invention relates to carbamate and urea derivatives of the formula I

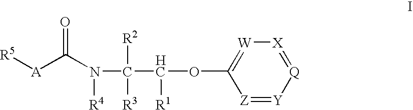

where R¹ is tetrazole or a group

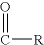

in which R has the following meanings:

a) a radical OR⁶, in which R⁶ is:

hydrogen, the cation of an alkali metal, the cation of an alkaline earth metal, a physiologically tolerable organic ammonium ion such as tertiary C₁-C₄-alkylamnonium or the ammonium ion;

C₃-C₈-cycloalkyl, C₁-C₈-alkyl, CH₂-phenyl, which can each be substituted by one or more radicals preferably by halogen, nitro, cyano, C₁-C₄-alkyl, C₁-C₄-haloalkyl, hydroxyl, C₁-C₄-alkoxy, mercapto, C₁-C₄-alkylthio, amino, NH(C₁-C₄-alkyl), N(C₁-C₄-alkyl)₂;

a C₂-C₆-alkenyl group or a C₃-C₆-alkynyl group, where these groups for their part can carry one to five halogen atoms; which may be substituted, preferably a phenyl radical R⁶ can furthermore be a phenyl radical which can carry one to five halogen atoms and/or one to three of the following radicals: nitro, cyano, C₁-C₄-alkyl, C₁-C₄-haloalkyl, hydroxyl, C₁-C₄-alkoxy, mercapto, C₁-C₄-alkylthio, amino, NH(C₁-C₄-alkyl), N(C₁-C₄-alkyl)₂;

b) pyrrolyl, pyrazolyl, imidazolyl and triazolyl, which can carry one or two halogen atoms, or one or two C₁-C₄-alkyl groups or one or two C₁-C₄-alkoxy groups;

c) a group

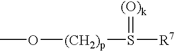

in which k assumes the values 0, 1 and 2, p the values 1, 2, 3 and 4 and R⁷ is

C₁-C₄-alkyl, C₃-C₈-cycloalkyl, C₂-C₆-alkenyl, C₂-C₆-alkynyl or phenyl, which can optionally be substituted one time or more, preferably by one to three of the following radicals: halogen, nitro, cyano, C₁-C₄-alkyl, C₁-C₄-haloalkyl, hydroxyl, C₁-C₄-alkoxy, C₁-C₄-alkylthio, mercapto, amino, NH(C₁-C₄-alkyl), N(C₁-C₄-alkyl)₂:

d) a radical

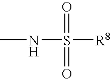

in which R⁸ is:

C₁-C₄-alkyl, C₂-C₆-alkenyl, C₂-C₆-alkynyl, C₃-C₈-cycloalkyl, where these radicals can carry a C₁-C₄-alkoxy radical, C₁-C₄-alkylthio radical and/or a phenyl radical as mentioned under c);

phenyl, substituted by one to three of the following radicals: halogen, nitro, cyano, C₁-C₄-alkyl, C₁-C₄-haloalkyl, hydroxyl, C₁-C₄-alkoxy, C₁-C₄-alkylthio, mercapto, amino, NH(C₁-C₄-alkyl), N(C₁-C₄-alkyl)₂.

The other substituents have the following meanings:

R² and R³ (which can be identical or different) are:

phenyl or naphthyl, each of which can be substituted by one or more of the following radicals: halogen, nitro, cyano, hydroxyl, mercapto, C₁-C₄-alkyl, C₂-C₄-alkenyl, C₂-C₄-alkynyl, C₁-C₄-haloalkyl, C₁-C₄-alkoxy, phenoxy, C₁-C₄-haloalkoxy, C₁-C₄-alkylthio, amino, NH(C₁-C₄-alkyl), N(C₁-C₄-alkyl)₂ or phenyl, which can be mono- or polysubstituted by halogen, nitro, cyano, C₁-C₄-alkyl, C₁-C₄-haloalkyl, C₁-C₄-alkoxy, C₁-C₄-haloalkoxy or C₁-C₄-alkylthio;

phenyl or naphthyl, which are bonded to one another in the ortho position via a direct bond, a methylene, ethylene or ethenylene group, an oxygen or sulfur atom or an SO₂—, NH— or N-alkyl group;

C₅-C₆-cycloalkyl;

R⁴ is hydrogen, C₁-C₄-alkyl;

R⁵ is C₁-C8-alkyl optionally substituted, preferably monosubstituted by halogen, hydroxyl, C₁-C₄-alkoxy or phenyl, which for its part can carry one to three of the following substituents: halogen, cyano, C₁-C₄-alkoxy, C₁-C₄-alkyl, C₁-C₄-alkylthio, NH(C₁-C₄-alkyl), N(C₁-C₄-alkyl)₂, amino, carboxyl;

C₃-C₈-cycloalkyl optionally substituted, preferably monosubstituted by: cyano, carboxyl, C₁-C₄-alkyl, C₁-C₄-haloalkyl, hydroxyl, C₁-C₄-alkoxy, amino, NH(C₁-C₄-alkyl), N(C₁-C₄-alkyl)₂;

phenyl or naphthyl, each of which can carry one to three of the following substituents: halogen, cyano, C₁-C₄-alkoxy, C₁-C₄-alkyl, C₁-C₄-alkylthio,NH(C₁-C₄-alkyl), N(C₁-C₄-alkyl)₂, amino or carboxyl;

or R⁵ forms a three- to seven-membered ring with NR⁹ as indicated under R⁹;

A is oxygen or NR9 where

R⁹ is hydrogen, C₁-C₄-alkyl;

or NR⁹ forms a three- to seven-membered saturated ring, which can be monosubstituted by C₁-C₄-alkyl and in which up to two of the methylene groups can be replaced by oxygen, sulfur, NH or N(C₁-C₄-alkyl), with R⁵ and an appropriate number of methylene groups;

w and Z (which can be identical or different) are:

nitrogen or methine; with the proviso that if W and Z=methine, then Q=nitrogen;

X is nitrogen or CR¹⁰;

Y is nitrogen or CR¹¹;

Q is nitrogen or CR¹²; with the proviso that if Q=nitrogen, then X=CR¹⁰ and Y=CR¹¹;

the proviso further applies that at most three of the ring members designated by W, X, Q, Y and Z are nitrogen;

R¹⁰ and R¹¹(which can be identical or different) are:

hydrogen, halogen, , C₁-C₄-haloalkoxy, C₃-C₆-alkenyloxy, C₃-C₆-alkynyloxy, C₁-C₄-alkylthio, C₁-C₄-alkylcarbonyl, C₁-C₄-alkoxycarbonyl, hydroxyl, NH₂, NH(C₁-C₄-alkyl), N(C₁-C₄-alkyl)₂, carboxyl;

C₁-C₄-alkyl, C₂-C₄-alkenyl, C₂-C₄-alkynyl, where these radicals can be mono- or polysubstituted by halog n, hydroxyl, mercapto, carboxyl, cyano, phenyl, C₁-C₄-alkoxy;

phenyl or phenoxy, each of which can be mono or disubstituted by halogen, nitro, cyano, C₁-C₄-alkyl, C₁-C₄-haloalkyl, C₁-C₄-alkoxy, C₁-C₄-haloalkoxy alkoxycarbonyl, alkylcarbonyl, amino;

C1-C4-alkyl, which may be mono- or polysubstituted, preferably by halogen, hydroxyl, carboxyl, cyano;

or CR¹⁰ or CR¹¹ is linked with CR¹² as indicated under R¹² to give a 5- or 6-membered ring;

R¹² is hydrogen, halogen, C₁-C₄-alkoxy, C₁-C₄-haloalkoxy, C₁-C₄-alkylthio, C₁-C₄-alkylcarbonyl, C₁-C₄-alkoxycarbonyl, NH(C₁-C₄-alkyl), N(C₁-C₄-alkyl)₂, hydroxyl, carboxyl, cyano, amino, mercapto;

C₁-C₄-alkyl, C₂-C₄-alkenyl, C₂-C₄-alkynyl, where these radicals can be mono- or polysubstituted by: halogen, hydroxyl, mercapto, carboxyl, cyano, amino, C₁-C₄-alkoxy;

or CR¹² forms a 5- or 6-membered alkylene or alkenylene ring, which can be substituted by one or two C₁-₄-alkyl groups, and in which one or more methylene groups in each case can be replaced by oxygen, sulfur, —NH or —N(C₁-C₄-alkyl), together with CR¹⁰ or CR¹¹.

The following definitions apply here and subsequently:

an alkali metal is, for example, lithium, sodium, potassium;

an alkaline earth metal is, for example, calcium, magnesium, barium;

organic ammonium ions are protonated amines such as, for example, ethanolamine, diethanolamine, ethylenediamine, diethylamine, triethylamine or piperazine;

C₃-C₈-cycloalkyl is, for example, cyclopropyl, cyclobutyl, cyclopentyl, cyclohexyl or cycloheptyl;

C₁-C₄-haloalkyl can be linear or branched such as, for example, fluoromethyl, difluoromethyl, trifluoromethyl, chlorodifluoromethyl, dichlorofluoromethyl, trichloromethyl, 1-fluoroethyl, 2-fluoroethyl, 2,2-difluoroethyl, 2,2,2-trifluoroethyl, 2-chloro-2,2-difluoroethyl, 2,2-dichloro-2-fluoroethyl, 2,2,2-trichloroethyl or pentafluoroethyl;

C₁-C₄-haloalkoxy can be linear or branched such as, for example, difluoromethoxy, trifluoromethoxy, chlorodifluoromethoxy, 1-fluoroethoxy, 2,2-difluoroethoxy, 1,1,2,2-tetrafluoroethoxy, 2,2,2-trifluoroethoxy, 2-chloro-1,1,2-trifluoroethoxy, 2-fluoroethoxy or pentafluoroethoxy;

C₁-C₈-alkyl can be linear or branched such as, for example, methyl, ethyl, 1-propyl, 2-propyl, 2-methyl-2-propyl, 2-methyl-1-propyl, 1-butyl, 2-butyl, n-hexyl, 3-methyl-1-pentyl, 4-methyl-2-pentyl, 3-methyl-2-hexyl, n-octyl;

C₂-C₆-alkenyl can be linear or branched such as, for example, ethenyl, 1-propen-3-yl, 1-propen-2-yl, 1-propen-1-yl, 2-methyl-1-propenyl, 1-butenyl, 2-butenyl, 1-penten-3-yl, 1-hexen-5-yl;

C₂-C₆-alkynyl can be linear or branched such as, for example, ethynyl, 1-propyn-1-yl, 1-propyn-3-yl, 1-butyn-4-yl, 2-butyn-4-yl or 1-hexyn-3-yl;

C₁-C₄-alkoxy can be linear or branched such as, for example, methoxy, ethoxy, propoxy, 1-methylethoxy, butoxy, 1-methylpropoxy, 2-methylpropoxy or 1,1-dimethylethoxy;

C₃-C₆-alkenyloxy can be linear or branched such as, for example, allyloxy, 2-buten-1-yloxy or 3-buten-2-yloxy;

C₃-C₆-alkynyloxy can be linear or branched such as, for example, 2-propyn-1-yloxy, 2-butyn-1-yloxy or 3-butyn-2-yloxy;

C₁-C₄-alkylthio can be linear or branched such as, for example, methylthio, ethylthio, propylthio, 1-methylethylthio, butylthio, 1-methylpropylthio, 2-methylpropylthio or 1,1-dimethylethylthio;

C₁-C₄-alkylcarbonyl can be linear or branched such as, for example, acetyl, ethylcarbonyl or 2-propylcarbonyl;

C₁-C₄-alkoxycarbonyl can be linear or branched such as, for example, methoxycarbonyl, ethoxycarbonyl, n-propoxycarbonyl, i-propoxycarbonyl or n-butoxycarbonyl;

halogen is, for example, fluorine, chlorine, bromine, iodine.

The invention further relates to those compounds from which the compounds of th formula I where R¹=COOH can be released (so-called prodrugs).

Preferred prodrugs are those in which the release proceeds under those conditions which prevail in certain body compartments, e.g. in the stomach, intestines, blood circulation, liver.

A preferred embodiment for “prodrugs” are those compounds in which the radical R¹ in formula (I) is present in masked form and the activation to give the “drug” produces a COOH function for R¹. The masking of certain chemical groups of a compound as a prodrug is a process familiar to the person skilled in the art (see, for example, “A Textbook of Drug Design and Development”, Krogsgard-Larsen and Bundgard, Harvwood Academic Publishers).

The invention further relates to the physiologically tolerable salts of compounds of the general formula I.

The compounds and also the intermediates for their preparation, such as, for example, II and III, can have one or more asymmetric substituted carbon atoms. Compounds of this type can be present as pure enantiomers or pure diastereomers or as a mixture thereof. The use of an enantiomerically pure compound as active compound is preferred. The invention further relates to the use of the abovementioned carbamate and urea derivatives for the production of drugs, in particular for the production of inhibitors for endothelin receptors.

The compounds having the general formula III can be prepared, as described in WO 98/58916, by reduction of the azido compounds of the general formula II. Alternatively to the reductants described there, trialkylphosphanes, for example tri(n-butyl)phosphane can also be employed to good effect.

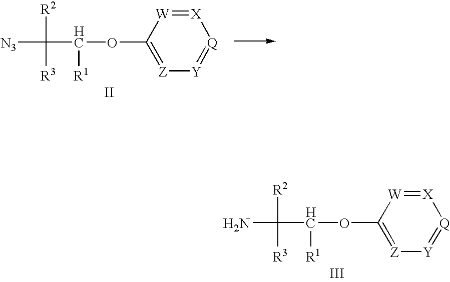

The compounds of the general formula I according to the invention in which A is oxygen (Ia) can be prepared, for example, by reacting the carboxylic acid derivatives of the general formula III with chloroformic acid esters of the general formula IV. To this end, the compounds mentioned are reacted in a molar ratio of approximately 1:1 to 1:5 in the presence of a base and of a suitable diluent. For this purpose, all solvents which are inert to the reagents used can be used.

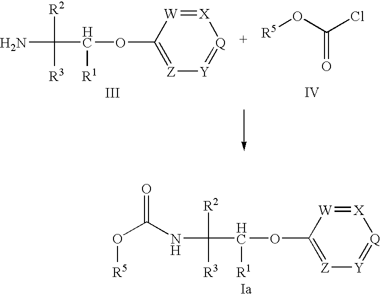

Examples of the abovementioned solvents or diluents are aliphatic, alicyclic and aromatic hydrocarbons, which can each optionally be chlorinated, such as, for example, hexane, cyclohexane, petroleum ether, ligroin, benzene, toluene, xylene, methylene chloride, chloroform, carbon tetrachloride, ethyl chloride and trichloroethylene, ethers, such as, for example, diisopropyl ether, dibutyl ether, methyl tert-butyl ether, dioxane and tetrahydrofuran, nitrites, such as, for example, acetonitrile and propionitrile, acid amides, such as, for example, dimethylformamide, dimethylacetamide and N-methylpyrrolidone, sulfoxides and sulfones, such as, for example., dimethyl sulfoxide and sulfolane.

Bases which can be employed are, for example, tertiary aliphatic amines, such as, for example, triethylamine, diisopropylethylamine, N-methylmorpholine or N-methylpyrrolidine, and also aromatic nitrogen compounds which are inert under the reaction conditions, such as pyridine.

The reaction is in this case preferably carried out in a temperature range between 0° C. and the boiling point of the solvent or solvent mixture.

The compounds according to'the invention of the general formula I in which A is NR⁹ (Ib) can be prepared, for example, by first converting the carboxylic acid derivatives of the general formula III using phosgene or an equivalent thereof in the presence of one of the abovementioned diluents into an isocyanate V and subsequently converting this by reaction with R⁵R⁹NH into the compounds of the general formula Ib according to the invention.

The second step is preferably carried out in the presence of one of the abovementioned bases and diluents.

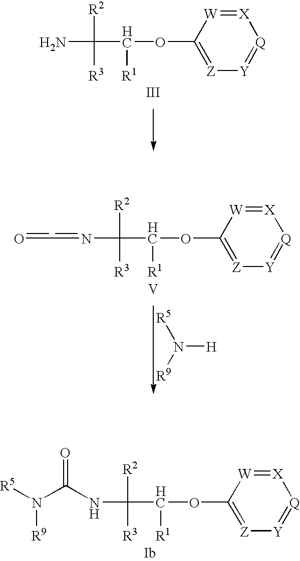

Both reaction steps are in this case preferably carried out in a temperature range between 0° C. and the boiling point of the solvent or solvent mixture.

If R¹ is an ester, the compounds where R¹=COOH can be prepared by acidic, basic or catalytic cleavage of the ester group.

Compounds of the type I where R¹=COOH can furthermore be directly obtained if an intermediate VI, in which R¹ is COOH, is deprotonated using three equivalents of a suitable base and reacted with compounds of the general formula VII. Here too, the reaction takes place in an inert solvent and in a temperature range from room temperature up to the boiling point of the solvent.

The base used in this reaction step can be an alkali metal or alkaline earth metal hydride such as sodium hydride, potassium hydride or calcium hydride, a carbonate such as alkali metal carbonate, e.g. sodium or potassium carbonate, an alkali metal or alkaline earth metal hydroxide such as sodium or potassium hydroxide, an organometallic compound such as butyllithium or an alkali metal amide such as lithium diisopropylamide.

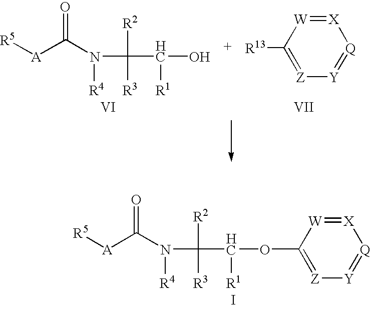

In formula VII, R¹³ is halogen or R¹⁴—SO₂—, where R¹⁴ can be C₁-C₄-alkyl, C₁-C₄-haloalkyl or phenyl, and the conditions mentioned at the outset apply for W, X, Q, Y and Z. The reaction preferably takes place in an inert solvent or diluent with addition of a suitable base, i.e. of a base which brings about deprotonation of the intermediate VI, in a temperature range from room temperature up to the boiling point of the solvent.

Compounds of the formula VII are known, in some cases commercially available or can be prepared in a generally known manner.

Compounds of the formula I can also be prepared by starting from the corresponding carboxylic acids, i.e. compounds of the formula I in which R¹ is COOH, and first converting these in a customary manner into an activated form such as an acid halide, an anhydride or imidazolide and then reacting this with an appropriate hydroxyl compound HOR⁶. This reaction can be carried out in the customary solvents and often necessitates the addition of a base, such as, for example, triethylamine, pyridine, imidazole or diazabicycloundecane being suitable. These two steps can also be simplified, for example, by allowing the carboxylic acid to act on the hydroxyl compound in the presence of a dehydrating agent such as a carbodiumide.

In addition, compounds of the formula I can also be prepared by starting from the salts of the corresponding carboxylic acids, i.e. from compounds of the formula I in which R¹ is a group COOM, where M can be an alkali metal cation or the equivalent of an alkaline earth metal cation. These salts can be reacted with many compounds of the formula R6-D, where D is a customary nucleofugic leaving group, for example halogen such as chlorine, bromine, iodine or aryl- or alkylsulfonyl optionally substituted by halogen, alkyl or haloalkyl, such as, for example, toluenesulfonyl and methylsulfonyl or another equivalent leaving group. Compounds of the formula R⁶-D having a reactive substituent D are known or can easily be obtained using the general specialized knowledge. This reaction can be carried out in the customary solvents and is advantageously formed with addition of a base, those mentioned above being suitable.

Compounds of the formula I in which R¹ is tetrazole can be prepared as described in WO 96/11914; further preparation procedures can be taken from the chemical technical literature and are found, for example, in Synthesis 767 (1993) and in J. Org. Chem. 56, 2395, (1991).

With respect to the biological action, carbamates and urea derivatives of the general formula I—both as pure enantiomers or as pure diastereomers or as a mixture thereof—are preferred in which the substituents have the following meanings:

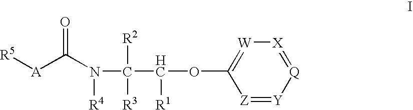

where R¹ is tetrazole or a group

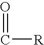

in which R has the following meanings:

a) a radical OR⁶, in which R⁶ is:

hydrogen, the cation of an alkali metal, the cation of an alkaline earth metal, a physiologically tolerable organic ammonium ion such as tertiary C₁-C₄-alkylammonium or the ammonium ion;

C₃-C₈-cycloalkyl, C₁-C₈-alkyl, CH₂-phenyl, which can each be substituted by one or more of the following radicals: halogen, C₁-C₄-alkyl, C₁-C₄-haloalkyl, hydroxyl, C₁-C₄-alkoxy;

a C₂-C₆-alkenyl group or a C₃-C₆-alkynyl group, where these groups for their part can carry one to five halogen atoms;

R⁶ can furthermore be a phenyl radical which can carry one to five halogen atoms and/or one to three of the following radicals: C₁-C₄-alkyl, C₁-C₄-haloalkyl, hydroxyl, C₁-C₄-alkoxy;

b) pyrrolyl, pyrazolyl, imidazolyl and triazolyl, which can carry one or two halogen atoms, or one or two C₁-C₄-alkyl groups or one or two C₁-C₄-alkoxy groups;

c) a group

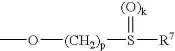

in which k assumes the values 0, 1 and 2, p the values 1, 2, 3 and 4 and R⁷ is

C₁-C₄-alkyl, C₃-C₈-cycloalkyl or phenyl, which can optionally be substituted by one to three of the following radicals: halogen, C₁-C₄-alkyl, C₁-C₄-haloalkyl, hydroxyl, C₁-C₄-alkoxy:

d) a radical

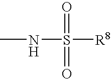

in which R⁸ is:

C₁-C₄-alkyl, C₃-CS-cycloalkyl, where these radicals can carry a C₁-C₄-alkoxy radical, C₁-C₄-alkylthio radical and/or a phenyl radical as mentioned under c);

phenyl, which can be substituted by one to three of the following radicals: halogen, C₁-C₄-alkyl, C₁-C₄-haloalkyl, hydroxyl, C₁-C₄-alkoxy.

The other substituents have the following meanings:

R² and R³ (which can be identical or different) are:

phenyl which can be substituted by one or more of the following radicals: halogen, nitro, cyano, hydroxyl, C₁-C₄-alkyl, C₁-C₄-haloalkyl, C₁-C₄-alkoxy, phenoxy, C₁-C₄-haloalkoxy, amino, NH(C₁-C₄-alkyl), N(C₁-C₄-alkyl)₂ or phenyl, which can be mono- or polysubstituted by halogen, C₁-C₄-alkyl, C₁-C₄-haloalkyl, C₁-C₄-alkoxy, C₁-C₄-haloalkoxy;

R⁴ is hydrogen, C₁-C₄-alkyl;

R⁵ is C₁-C₈-alkyl which can be monosubstituted by halogen, hydroxyl, C₁-C₄-alkoxy or phenyl, which for its part can carry one to three of the following substituents: halogen, cyano, C₁-C₄-alkoxy, C₁-C₄-alkyl carboxyl;

C₃-C₈-cycloalkyl which can be monosubstituted by: carboxyl, C₁-C₄-alkyl, hydroxyl, C₁-C₄-alkoxy;

phenyl or naphthyl, each of which can carry one to three of the following substituents: halogen, C₁-C₄-alkoxy, C₁-C₄-alkyl or carboxyl;

or R⁵ forms a three- to seven-membered ring with NR⁹ as indicated under R⁹;

A is oxygen or NR⁹ where

R⁹ is hydrogen, C₁-C₄-alkyl;

or NR9 forms a three- to seven-membered saturated ring, which can be monosubstituted by C₁-C₄-alkyl and in which up to two of the methylene groups can be replaced by oxygen with R⁵ and an appropriate number of methylene groups;

W and Z (which can be identical or different) are:

nitrogen or methine; with the proviso that if W and Z=methine, then Q=nitrogen;

X is nitrogen or CR¹⁰;

Y is nitrogen or CR¹¹;

Q is nitrogen or CR¹²; with the proviso that if Q=nitrogen, then X=CR¹⁰ and Y=CR¹¹;

the further proviso applies that at most three of the ring members designated by W, X, Q, Y and z are nitrogen;

R¹⁰ and R¹¹(which can be identical or different) are:

hydrogen, halogen, C₁-C₄-alkoxy, C₁-C₄-alkylcarbonyl, C₁-C₄-alkoxycarbonyl, hydroxyl, NH₂, NH(C₁-C₄-alkyl), N(C₁-C₄-alkyl)₂, carboxyl;

C₁-C₄-alkyl, which can be mono- or polysubstituted by halogen, hydroxyl, mercapto, carboxyl, phenyl, C₁-C₄-alkoxy; or phenoxy phenyl which can be mono- or disubstituted by halogen, C₁-C₄-alkyl, C₁-C₄-alkoxy, C₁-C₄-haloalkoxy, alkoxycarbonyl

or CR¹⁰ or CR¹¹ is linked with CR¹² as indicated under R¹² to give a 5- or 6-membered ring;

R¹² is hydrogen, halogen, C₁-C₄-alkoxy, NH(C₁-C₄-alkyl), N(C₁-C₄-alkyl)₂, hydroxyl, carboxyl, amino;

C₁-C₄-alkyl, which can be mono- or polysubstituted by: hydroxyl, carboxyl, amino, C₁-C₄-alkoxy;

or CR¹² forms a 5- or 6-membered alkylene or alkenylene ring, which can be substituted by one or two C₁₋₄-alkyl groups, and in which one or more methylene groups in each case can be replaced by oxygen, —NH or —N(C₁-C₄-alkyl), together with CR¹⁰ or CR¹¹.

Particularly preferred compounds of the formula I—both as pure enantiomers or pure diastereomers or as a mixture thereof—are those in which the substituents have the following meanings:

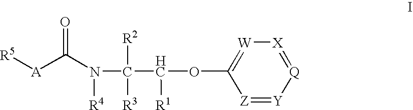

where R1 is tetrazole or a group

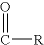

in which R has the following meanings:

a) a radical OR⁶, in which R⁶ is:

hydrogen, the cation of an alkali metal, the cation of an alkaline earth metal, a physiologically tolerable organic ammonium ion such as tertiary C₁-C₄-alkylammonium or the ammonium ion;

C₃-C₈-cycloalkyl, C₁-C₈-alkyl, CH₂-phenyl, which can each be substituted by one or more of the following radicals: halogen, C₁-C₄-alkyl, C₁-C₄-haloalkyl, hydroxyl, C₁-C₄-alkoxy;

R6 can furthermore be a phenyl radical which can carry one to five halogen atoms and/or one to three of the following radicals: C₁-C₄-alkyl, C₁-C₄-alkoxy;

b) a radical

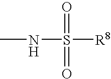

in which RB is:

C₁-C₄-alkyl, C₅-C₆-cycloalkyl; phenyl which can be substituted by one to three of the following radicals: halogen, C₁-C₄-alkyl, C₂-C₄-alkoxy.

The other substituents have the following meanings:

R² and R³ (which can be identical or different) are:

phenyl which can be substituted by one or more of the following radicals: halogen, C₁-C₄-alkyl, C₁-C₄-alkoxy;

R⁴ is hydrogen:

R⁵ is C₁-C₄-alkyl which can be monosubstituted by hydroxyl, C₁-C₄-alkoxy or phenyl, which for its part can carry one to three of the following substituents: halogen, C₁-C₄-alkoxy or C₁-C₄-alkyl;

C₅-C₆-cycloalkyl, which can carry a C₁-C₄-alkyl group;

phenyl which can carry one to three of the following substituents: halogen, C₁-C₄-alkoxy or C₁-C₄-alkyl;

or R⁵ forms one of the cyclic groups indicated under R⁹ with NR⁹;

A is oxygen or NR⁹ where

R⁹ is hydrogen, C₁-C₄-alkyl;

or NR⁹ forms a pyrrolidinyl, piperidinyl, morpholinyl or N-methylpiperazinyl radical with R⁵;

W is nitrogen;

X is CR¹⁰;

Y is CR¹¹;

Z is nitrogen or methine;

Q is nitrogen or CR¹²;

R¹⁰ and R¹¹(which can be identical or different) are:

hydrogen, halogen, C₁-C₄-alkoxy;

C₁-C₄-alkyl which can be monosubstituted by hydroxyl or carboxyl;

trifluoromethyl;

or phenoxy, phenyl which can be mono- or disubstituted by halogen, C₁-C₄-alkyl, C₁-C₄-alkoxy, C₁-C₄-haloalkoxy alkoxycarbonyl

or CR¹⁰ or CR¹¹ is linked with CR¹² as indicated under R¹² to give a 5- or 6-membered ring;

R¹² is hydrogen, halogen, C₁-C₄-alkoxy;

C₁-C₄-alkyl which can be monosubstituted by hydroxyl;

or CR¹² forms a 5- or 6-membered alkylene or alkenylene ring, which can be substituted by one or two C₁₋₄-alkyl groups, and in which a methylene group can be replaced by oxygen, together with CR¹⁰ or CR¹¹.

The compounds of the present invention offer a novel therapeutic potential for the treatment of hypertension, pulmonary hypertension, myocardial infarct, angina pectoris, arrhythmia, acute/chronic kidney failure, chronic cardiac insufficiency, renal insufficiency, cerebral vasospasms, cerebral ischemia, subarachnoid hemorrhages, migraine, asthma, atherosclerosis, endotoxic shock, endotoxin-induced organ failure, intravascular coagulation, restenosis after angioplasty and bypass operations, benign prostate hyperplasia, impaired erection, glaucoma, kidney failure or hypertension, which is ischemic and caused by intoxication, metastasization and growth of mesenchymal tumors, cirrhosis of the liver, contrast agent-induced kidney failure, pancreatitis, gastrointestinal ulcers.

A further subject of the invention is combinations of endothelin receptor antagonists of the formula I and inhibitors of the renin-angiotensin system. Inhibitors of the renin-angiotensin system are renin inhibitors, angiotensin II antagonists and angiotensin-converting-enzyme (ACE) inhibitors. Combinations of endothelin receptor antagonists of the formula I and ACE inhibitors are preferred.

A further subject of the invention is combinations of endothelin receptor antagonists of the formula I and beta-blockers.

A further subject of the invention is combinations of endothelin receptor antagonists of the formula I and diuretics.

A further subject of the invention is combinations of endothelin receptor antagonists of the formula I and calcium antagonists.

A further subject of the invention is combinations of endothelin receptor antagonists of the formula I and substances which block the action of VEGF (vascular endothelial growth factor). Substances of this type are, for example, antibodies directed against VEGF or specific binding proteins or alternatively low-molecular-weight substances which can specifically inhibit VEGF release or receptor binding.

The abovementioned combinations can be administered simultaneously or sequentially. They can be employed both in a single pharmaceutical formulation or alternatively in separate formulations. The administration form can also be different, for example, the endothelin receptor antagonists can be administered orally and VEGF inhibitors parenterally.

These combination preparations are especially suitable for the treatment and prevention of hypertension and its sequelae, and for the treatment of cardiac insufficiency.

The good action of the compounds can be shown in the following experiments:

Receptor Binding Studies

For binding studies, cloned human ET_{A} or ET_{B} receptor-expressing CHO cells were employed.

Membrane Preparation

The ET_{A} or ET_{B} receptor-expressing CHO cells were proliferated in DMEM NUT MIX F₁₂ medium (Gibco, No. 21331-020) using 10% fetal calf serum (PAA Laboratories GmbH, Linz, No. A15-022), 1 mM glutamine (Gibco No. 25030-024), 100 U/ml of penicillin and 100 μg/ml of streptomycin (Gibco, Sigma No P-0781). After 48 hours, the cells were washed with PBS and incubated at 37° C. for 5 minutes with 0.05% trypsin-containing PBS. Neutralization was then carried out with medium and the cells were collected by centrifugation at 300×g.

For membrane preparation, the cells were adjusted to a concentration of 10⁸ cells/ml of buffer (50 mM tris-HCl buff r, pH 7.4) and then disintegrated by ultrasound (Branson Sonifier 250, 40-70 seconds/constant/output 20).

Binding Tests

For the ET_{A} and ET_{B} receptor binding test, the membranes were suspended in incubation buffer (50 mM tris HCl, pH 7.4 with 5 mM MnCl₂, 40 mg/ml of bacitracin and 0.2% of BSA) in a concentration of 50 μg of protein per test batch and incubated at 25° C. with 25 pM of [125J]-ET₁ (ET_{A} receptor test) or 25 pM of [125I)-ET₃ (ET_{B} receptor test) in the presence and absence of test substance. The nonspecific binding was determined using 10⁻⁷ M ET₁. After 30 min, the free and the bound radioligand was separated by filtration through GF/B glass fiber filters (Whatman, England) on a Skatron cell collector (Skatron, Lier, Norway) and the filters were washed with ice-cold tris HCl buffer, pH 7.4 with 0.2% of BSA. The radioactivity collected on the filters was quantified using a Packard 2200 CA liquid scintillation counter.

Functional vessel test for endothelin receptor antagonists A K⁺ contracture was first induced in rabbit aortal segments after a pretension of 2 g and a relaxation time of 1 h in Krebs-Henseleit solution at 37° C. and a pH of between 7.3 and 7.4. After washing-out, an endothelin dose-response curve is plotted up to the maximum.

Potential endothelin antagonists are applied to other preparations in the same vessel 15 min before the start of the endothelin dose-response curve. The effects of the endothelin are calculated in % of the K⁺ contracture. If the endothelin antagonists are active, there is a shift to the right of the endothelin dose-response curve.

Testing of the ET Antagonists In Vivo:

Male SD rats 250-300 g in weight were anaesthetized with aminobarbital, artificially ventilated, vagotomized and pithed. The carotid artery and jugular vein were catheterized.

In control animals, the intravenous administration of 1 μg/kg of ET-1 leads to a marked blood pressure increase, which lasts for a relatively long period of time.

The test compounds were injected i.v. (1 ml/kg) into the test animals 30 min before the ET-1 administration. For the determination of the ET-antagonist properties, the blood pressure changes in the test animals were compared with those-in the control animals.

P.O. Testing of the ET Antagonists:

Male normotonic rats (Sprague Dawley, Janvier) 250 to 350 g in weight are orally pretreated with the test substances. 80 minutes later, the animals are anaesthetized with urethane and the carotid artery (for blood pressure measurements) and also the jugular vein (administration of big endothelin/endothelin-1) are catheterized.

After a stabilization phase, big endothelin (20 μg/kg, admin. vol. 0.5 ml/kg) or ET-1 (0.3 μg/kg, admin. vol. 0.5 ml/kg) is given intravenously. Blood pressure and heart rates are recorded continuously for 30 minutes. The marked and long-lasting blood-pressure changes are calculated as the area under the curve (AUC). For the determination of the antagonistic action of the test substances, the AUC of the substance-treated animals is compared with the AUC of the control animals.

The compounds according to the invention can be administered orally or parenterally (subcutaneously, intravenously, intramuscularly, intraperitonealy) in a customary manner. Administration can also be carried out through the nasopharynx using vapors or sprays.

The dose depends on the age, condition and weight of the patient and on the manner of administration. As a rule, the daily dose of active compound is between approximately 0.5 and 50 mg/kg of body weight in the case of oral administration and between approximately 0.1 and 10 mg/kg of body weight in the case of parenteral administration.

The novel compounds can be administered in liquid or solid form in the customary pharmaceutical administration forms, e.g. as tablets, film-coated tablets, capsules, powders, granules, coated tablets, suppositories, solutions, ointments, creams or sprays. These are produced in the customary manner. The active compounds can in this case be processed using the customary pharmaceutical excipients such as tablet binders, fillers, preservatives, tablet disintegrants, flow regulators, plasticizers, wetting agents, dispersants, emulsifiers, solvents, release-delaying agents, antioxidants and/or propellants (cf. H. Sucker et al.:

Pharmazeutische Technologie, Thieme-Verlag, Stuttgart, 1991). The administration forms thus obtained normally contain the active compound in an amount from 0.1 to 90% by weight.

### SYNTHESIS EXAMPLES

### Example 1

Methyl 3-azido-2-[(4,6-dimethoxy-2-pyrimidinyl)oxy]-3,3-diphenyl Propionate

A solution of methyl 3-azido-2-hydroxy-3,3-diphenyl propionate (4.47 g; 15.0 mmol) and 4,6-dimethoxy-2-methylsulfonylpyrimidine (3.61 g; 16.5 mmol) in anhydrous dimethylformamide (20 ml) was treated with potassium carbonate (1.04 g; 7.52 mmol) and stirred at 80° C. for 3 hours. After cooling, the mixture was stirred at room temperature for a further 2 days. The batch was poured onto ice water (100 ml) and extracted with ethyl acetate (3×). The combined organic extracts were dried over magnesium sulfate and evaporated in vacuo. The residual oil (8.30 g) was employed further without further purification.

¹H-NMR (270 MHz, CDCl₃): 7.2-7.5 (m, 10H); 6.0 (s, 1H); 5.7 (s, 1 H); 3.8 (s, 6 H); 3.4 (s, 3 H).

HPLC-MSD: M+H⁺=436.

### Example 2

Methyl 3-amino-2-[(4,6-dimethoxy-2-pyrimidinyl)oxy]-3,3-diphenyl Propionate

Methyl 3-azido-2-[(4,6-dimethoxy-2-pyrimidinyl)oxy]-3,3-diphenyl propionate (8.30 g; crude) was dissolved in a mixture of methanol (50 ml) and dichloromethane (30 ml), treated with a 10% strength palladium/activated carbon hydrogenation catalyst (about 1 g) and stirred at room temperature for 20 hours under a hydrogen atmosphere. The catalyst was then filtered off and the filtrate was concentrated. Precipitation from dichloromethane/hexane afforded the desired compound as a colorless solid (3.50 g; 8.55 mmol, 57% over 2 stages).

¹H-NMR (270 MHz, CDCl₃): 7.2-7.6 (m, 10 H); 5.9 (s, 1 H); 5.7 (s, 1 H); 3.9 (s, 6 H); 3.2 (s, 3 H); 2.0-2.6 (s br, 2 H).

### Example 3

Methyl 3-[(benzyloxycarbonyl)amino]-2-[(4,6-dim thoxy-2-pyrimidinyl)oxy]-3,3-diphenyl Propionate

A solution of methyl 3-amino-2-[(4,6-dimethoxy-2-pyrimidinyl)oxy]-3,3-diphenyl propionate (300 mg; 730 μmol) in anhydrous dichloromethane (10 ml) was treated with benzyl chloroformate (115 μl; 810 μmol) and pyridine (150 μl; 1.83 mmol) and stirred at room temperature. After 2 hours, benzyl chloroformate (115 μl, 810 μmol) and pyridine (150 μl, 1.83 mmol) were added and the batch was stirred for two further hours. In order to complete the reaction, it was treated once more with benzyl chloroformate (115 μl) and pyridine (150 μl), stirred for 30 minutes and the reaction was discontinued by shaking the reaction mixture with saturated sodium hydrogencarbonate solution. The organic phase was washed with aqueous citric acid, dried over magnesium sulfate and concentrated. The residue which remained was purified by column chromatography; 280 mg (500 μmol with 96% purity, 68% yield) of the title compound were obtained.

HPLC-MSD: M+H⁺=544.

### Example 4 (I-140)

N-[(Benzyloxy)carbonyl]-2-[(4,6-dimethoxy-2-pyrimidinyl)oxy]-3,3-diphenyl-β-alanine

A solution of methyl 3-[(benzyloxycarbonyl)amino]-2[(4,6-dimethoxy-2-pyrimidinyl)oxy]-3,3-diphenyl propionate (280 mg, 550 μmol with 96% purity) in a mixture of water (5 ml) and tetrahydrofuran (10 ml) was-treated with lithium hydroxide (25 mg, 1.04 mmol) and stirred at room temperature for 24 hours. The batch was diluted with water and extracted with ether; the aqueous phase was acidified with citric acid and again extracted with ether. As the target compound was contained in both organic extracts, these were purified, dried using magnesium sulfate and evaporated in vacuo. Precipitation from dichloromethane/n-hexane afforded the desired product in 95% purity as a colorless solid (100 mg, 180 μmol, 36% yield).

¹H-NMR (360 MHz, CDCl₃): 7.5 (m, 2 H); 7.1-7.4 (m, 13 H); 6.4 (s, 2 H); 5.7 (s, 1 H); 5.0 (s, 2 H); 3.8 (s, 6 H).

ESI-MS: M⁺=529.

### Example 5

Benzyl 3-azido-2-hydroxy-3,3-diphenyl Propionate

A solution of methyl 3-azido-2-hydroxy-3,3-diphenyl propionate (2.00 g, 6.73 mmol) in water (10 ml) and tetrahydrofuran (20 ml) was treated with 1-molar sodium hydroxide solution (10.0 ml) and stirred at room temperature for three hours. After diluting with water and acidifying with dilute hydrochloric acid, the batch was extracted with ether. The organic extracts were dried over magnesium sulfate and evaporated in vacuo. The residual crude oil (2.42 g) was taken up in dimethylformamide (30 ml) without further purification and treated with potassium carbonate (2.79 g; 20.2 mmol). After stirring at room temperature for 15 [lacuna], benzyl bromide (1.21 g; 0.84 ml; 7.07 mmol) was added dropwise and the batch was subsequently stirred at room temperature for 16 hours. It was then diluted with water, acidified with aqueous citric acid and extracted with ether. The combined ethereal extracts were backwashed with water, dried over magnesium sulfate and evaporated in vacuo. The residue was crystallized from ether and yielded 1.63 g (4.28 mmol; 64% yield) of the pure title compound.

HPLC-MSD: M+K⁺=412.

### Example 6

Benzyl 3-azido-2-[(4,6-dimethoxy-2-pyrimidyl)oxy]-3,3-diphenyl Propionate

A mixture of benzyl 3-azido-2-hydroxy-3,3-diphenyl propionate (1.62 g; 4.28 mmol) and potassium carbonate (296 mg; 2.14 mmol) in dimethylformamide (15 ml) was treated with 4,6-dimethoxy-2-methylsulfonylpyrimidine (1.03 g; 4.71 mmol) and stirred at 80° C. for one hour. The mixture was subsequently stirred at room temperature for two days and the reaction was then terminated by dilution. The mixture was extracted with ethyl acetate and the combined organic extracts were washed with water, dried over magnesium sulfate and evaporated in vacuo. The crude residue (2.27 g; 4.26 mmol with 96% purity according to HPLC, 99% yield) was reacted further without further purification.

HPLC-MSD: M+H⁺=512.

### Example 7

Benzyl 3-amino-2-[(4,6-dimethoxy-2-pyrimidyl)oxy]-3,3-diph nyl Propionate

Tri(n-butyl)phosphine (492 mg, 2.43 mmol) was added to a solution of benzyl 3-azido-2-[(4,6-dimethoxy-2-pyrimidyl)oxy]-3,3-diphenyl propionate (1.18 g; 2.21 mmol with 96% purity) in a 2:1 mixture of ether and dichloromethane (30 ml). After stirring at room temperature for one hour, the reaction mixture was extracted three times with aqueous citric acid. The organic phase was dried over magnesium sulfate and evaporated in vacuo. The residue (1.75 g) was purified by column chromatography. 930 mg (1.92 mmol, 87% yield) of the pure amine were obtained.

¹H-NMR (270 MHz, CDCl₃): 7.4-7.5 (m, 4 H); 7.2-7.3 (m, 9 H); 6.9 (m, 2 H); 6.0 (s, 1 H); 5.7 (s, 1 H); 4.9 (d, 1 H); 4.5 (d, 1 E); 3.8 (s, 6 H); 2.4 (s br, 2 H).

ESI-MS: M⁺=485.

### Example 8

Benzyl 2-[(4,6-dimethoxy-2-pyrimidinyl)oxy]-3-{[(4-methoxyphenoxy)carbonyl]amino}-3,3-diphenyl Propionate

Benzyl 3-amino-2-[(4,6-dimethoxy-2-pyrimidyl)oxy]-3,3-diphenyl propionate (368 mg, 0.76 mmol) was initially introduced into anhydrous dichloromethane (10 ml) with a spatula tipful of N,N-dimethylaminopyridine and treated in succession with pyridine (150 mg, 1.89 mmol) and 4-methoxyphenyl chloroformate (212 mg; 1.14 mmol). The batch was stirred at room temperature for 30 minutes. After termination of the reaction by shaking with sodium hydrogencarbonate solution, the organic phase was diluted with ether, washed three times with aqueous citric acid, dried over magnesium sulfate and evaporated in vacuo. The residue was purified by column chromatography; 286 mg (0.42 mmol with 94% purity, 56% yield) of the target compound were obtained.

HPLC-MSD: M+H⁺=636.

### Example 9 (I-170)

2-[(4,6-Dimethoxy-2-pyrimidinyl)oxy]-N-[(4-methoxyphenoxy)-carbonyl]-3,3-diphenyl-β-alanine

A solution of benzyl 2-[(4,6-dimethoxy-2-pyrimidinyl)oxy]-3-{[(4-methoxyphenoxy)carbonyl]amino}-3,3-diphenyl propionate (286 mg, 0.42 mmol) in ethyl acetate (10 ml) was treated with a spatula tipful of a 10% strength palladium/activated carbon hydrogenation catalyst and stirred under a hydrogen atmosphere for one hour. The catalyst was then filtered off and the filtrate was concentrated in vacuo. Crystallization from dichloromethane/n-hexane yielded the pure carboxylic acid (140 mg, 0.26 nmol, 61% yield).

¹H-NMR (360 MHz, CDCl₃): 7.5-7.6 (d, 2 H); 7.2-7.4 (m, 10 H); 6.9 (s br, 1 B); 6.8 (d, 2 H); 6.6 (s, 1 H); 6.4 (s, 1 H); 5.7 (s, 1 H); 3.9 (s, 6 H); 3.7 (s, 3 H).

ESI-MS: M⁺=545.

### Example 10

Methyl 2-[(4,6-dimethoxy-2-pyrimidinyl)oxy]-3-({[(4-methoxybenzyl)(methyl)amino]carbonyl}amino)-3,3-diphenyl Propionate

A solution of methyl 3-amino-2-[(4,6-dimethoxy-2-pyrimidinyl)oxy]-3,3-diphenyl propionate (300 mg, 0.71 mmol, see Example 2) in 1,4-dioxane (10 ml) was treated with triphosgene (222 mg, 0.75 mmol) and stirred at 80° C. for one hour. After cooling to room temperature, a solution of N-ethyldiisopropylamine (0.31 ml, 1.78 mmol) and N-(4-methoxybenzyl)-N-methylamine (124 mg, 0.82 mmol) in 1,4-dioxane (2 ml) was added dropwise. The batch was stirred at 70° C. again for two hours and then concentrated on a rotary evaporator. The residue was taken up in ethyl acetate and washed with saturated sodium chloride solution. The organic phase was dried over magnesium sulfate and evaporated in vacuo. The residue was taken up in dichloromethane and the product was precipitated by addition of hexane. 443 mg of a colorless solid were obtained (0.69 mmol with 91% purity, 97% yield).

HPLC-MSD: M+H⁺=587.

### Example 11 (I-23)

2-[(4,6-Dimethoxy-2-pyrimidinyl) oxy]-N-{[(4-methoxybenzyl)-(methyl)amino]carbonyl}-3,3-diphenyl-β-alanine

A solution of methyl 2-[(4,6-dimethoxy-2-pyrimidinyl)oxy]-3-({[(4-methoxybenzyl)(methyl)amino]carbonyl}amino)-3,3-diphenyl propionate (443 mg, 0.69 mmol with 91% purity) was initially introduced into a mixture of tetrahydrofuran (10 ml) and water (5 ml) and treated with 1-molar sodium hydroxide solution (1.03 ml). The mixture was stirred at room temperature for 16 hours and then at 40° C. for a further two hours to complete the reaction. The reaction mixture was then diluted with water, acidified with citric acid and extracted with ether. The organic extracts were backwashed with water, dried over magnesium sulfate and evaporated in vacuo. The residue was lyophilized; 290 mg (0.49 mmol, 72% yield) of the title compound were obtained.

¹H-NMR (360 MHz, CDCl₃): 7.2-7.4 (m, 10 H); 6.9 (d, 2 H); 6.8 (m, 3 H); 5.7 (s, 1 H); 5.6 (s br, 1 H); 4.5 (d, 1 H); 4.3 (d, 1 H); 3.9 (s, 6 H); 3.8 (s, 3 H); 2.9 (s, 3 H).

ESI-MS: M⁺=572.

The following were prepared analogously to Example 4:

### Example 12 (I-230)

2-[(4,6-Dimethoxy-2-pyrimidinyl)oxy]-N-(methoxycarbonyl)-3,3-diphenyl-β-alanine

¹H-NMR (400 MHz, d₆-DMSO): 8.1 (s br, 1 H); 7.5 (m app d, 2H); 7.1-7.4 (m, 8H); 6.4 (s br, 1H); 5.8 (s, 1H); 3.8 (s, 6 H); 3.4 (s, 3 H).

¹³C-NMR (100 MHz, d₆-DMSO): 172.2 (s); 169.8 (s); 163.0 (s); 154.8 (s); 142.3 (s); 140.0 (s); 128.8 (d); 128.1 (d); 127.4 (d); 127.2 (d); 127.03 (d); 126.99 (d); 126.6 (d); 83.0 (d); 77.2 (d); 65.1 (s); 54.1 (q), 51.1 (q).

ESI-MS: M⁺=453.

### Example 13 (I-266)

N-{[(4,5-Dimethoxy-2-nitrobenzyl)oxy]carbonyl}-2-((4,6-dimethoxy-2-pyrimidinyl)oxy]-3,3-diphenyl-β-alanine

¹H-NMR (360 MHz, CDCl₃): 7.7 (s, 1 H); 7.6 (m, 2 H); 7.2-7.4 (m, 8 H); 6.9 (s br, 1 H); 6.6 (s, 1 H); 6.4 (s, 1 H); 5.7 (s, 1 H); 5.4 (s br, 2 H); 3.9 (s, 3 H); 3.8 (s, 9 H).

ESI-MS: M⁺=634.

The following was prepared analogously to Example 9:

### Example 14 (I-168)

2-[(4,6-Dimethoxy-2-pyrimidinyl)oxy]-N-[(3,4-dimethylphenoxy)-carbonyl]-3,3-diphenyl-β-alanine

¹H-NMR (360 MHz, CDCl₃): 7.5-7.6 (m app d, 2 H); 7.2-7.4 (m, 8 H);

7.0 (d, 1 H); 6.7 (s br, 2 H); 6.6 (s, 1 H); 6.4 (s, 1 H); 5.7 (s, 1 H); 3.8 (s, 6 H); 2.2 (s, 6 H).

ESI-MS: M⁺=543.

The following were prepared analogously to Example 11:

### Example 15 (I-86)

N-[(Diethylamino)carbonyl]-2-[(4,6-dimethoxy-2-pyrimidinyl)oxy]-3,3-diphenyl-β-alanine

¹H-NMR (360 MHz, CDCl₃): 7.3-7.5 (m, 10 H); 6.8 (s, 1 H); 5.7 (s, 1 H); 5.4 (s br, 1 H); 3.9 (s, 6 H); 3.2-3.4 (m, 4 H); 1.1 (t, 6 H).

ESI-MS: M⁺=494.

### Example 16 (I-260)

N-{[Benzyl(methyl)amino]carbonyl}-2-[(4,6-dimethoxy-2-pyrimidinyl)oxy]-3,3-diphenyl-β-alanine

¹H-NMR (360 MHz, CDCl₃): 7.2-7.4 (m, 13 H); 7.0-7.1 (m, 2 H); 6.9 (s, 1 H); 5.7 (s, 1 H); 5.6 (s br, 1 H); 4.6 (d, 1 H); 4.3 (d, 1 H); 3.9 (s, 6 H); 2.9 (s, 3 H).

ESI-MS: M⁺=542.

### Example 17 (I-155)

2-[(4,6-Dimethoxy-2-pyrimidinyl)oxy]-N-[(methylanilino)carbonyl]-3,3-diphenyl-β-alanine

¹H-NMR (360 MHz, CDCl₃): 7.1-7.5 (m, 15 H); 7.0 (s, 1 H); 5.7 (s, 1 H); 5.3 (s, 1 H); 3.9 (s, 6 H); 3.2 (s, 3 H).

ESI-MS: M⁺=528.

### Example 18 (I-11)

2-[(4,6-Dimethoxy-2-pyrimidinyl)oxy]-N-[(dimethylamino)carbonyl]-3,3-diphenyl-β-alanine

¹H-NMR (360 MHz, CDCl₃): 7.7 (m app d, 2 H); 7.2-7.5 (m, 8 H); 6.6 (s, 1 H); 6.2 (s, 1 H), 5.7 (s, 1 H); 3.8 (s, 6 H); 3.0 (s, 6 H).

ESI-MS: M⁺=466.

### Example 19 (I-121)

2-[(4,6-Dimethoxy-2-pyrimidinyl)oxy]-N-{[4-methoxy(methyl)-anilino]carbonyl}-3,3-diphenyl-β-alanine

¹H-NMR (360 MHz, CDCl₃): 7.3-7.4 (m, 3 H); 7.15-7.3 (m, 7 H); 7.1 (m, 2 H); 7.0 (m, 3 H); 6.8 (d, 2 H); 5.7 (s, 1 H); 5.3 (s br, 1 H); 3.9 (s, 6 H); 3.8 (s, 3 H); 3.2 (s, 3 H).

ESI-MS: M⁺=558.

### Example 20 (I-194)

2-[(4,6-Dirnethyl-2-pyrimidinyl)oxy]-N-{[(4-methoxybenzyl)-(methyl)amino]carbonyl}-3,3-diphenyl-β-alanine

¹H-NMR (360 MHz, CDCl₃): 7.2-7.4 (m, 10 H); 6.9-7.0 (m, 3 H), 6.8 (d, 2 H); 6.7 (s, 1 H), 5.7 (s br, 1 H); 4.5 (d, 1 H); 4.3 (d, 1 H); 3.8 (s, 3 H); 2.8 (s, 3 H), 2.3 (s, 6 H).

ESI-MS: M⁺=540.

### Example 21 (I-75)

2-[(4,6-Dimethyl-2-pyrimidinyl)oxy]-N-(4-morpholinylcarbonyl)-3,3-diphenyl-β-alanine

¹H-NMR (360 MHz, CDCl₃): 7.5 (d, 2 H); 7.2-7.4 (m, 8 H); 6.7 (s, 1 H); 6.6 (s, 1 H); 6.1 (s br, 1 H); 3.5-3.7 (m, 4 H); 3.3-3.5 (m, 2 H); 3.2-3.3 (m, 2 H); 2.3 (s, 6 H).

ESI-MS: M⁺=476.

### Example 22 (I-52)

2-[(4,6-Dimethyl-2-pyrimidinyl)oxy]-3,3-diphenyl-N-(1-piperidinylcarbonyl)-β-alanine

¹H-NMR (360 MHz, CDCl₃): 7.5 (d, 2 H); 7.2-7.4 (m, 8 H); 6.9 (s, 1 H); 6.6 (s, 1 H); 5.7 (s br, 1 H); 3.3-3.5 (m, 2 H); 3.2-3.3 (m, 2 H); 2.3 (s, 6 H); 1.3-1.7 (m, 6 H).

ESI-MS: M⁺=474.

### Example 23 (I-26)

2-[(4,6-Dimethyl-2-pyrimidinyl)oxy]-3,3-diphenyl-N-(1-pyrrolidinylcarbonyl)-β-alanine

¹H-NMR (360 MHz, CDCl₃): 7.5 (m, 2 H); 7.2-7.4 (m, 8 H); 7.0 (s, 1 H); 6.7 (s, 1 H); 5.3 (s br, 1 H); 3.3-3.4 (m, 4 H); 2.3 (s, 6 H); 1.8-2.0 (m, 4 H).

ESI-MS: M⁺=460.

### Example 24 (I-202)

N-{[Cyclohexyl(methyl)amino]carbonyl}-2-[(4,6-dimethyl-2-pyrimidinyl)oxy]-3,3-diphenyl-β-alanine

¹H-NMR (400 MHz, CDCl₃): 7.5 (d, 2 H); 7.2-7.4 (m, 8 H); 6.8 (s, 1 H); 6.6 (s, 1 H); 6.1 (s br, 1 H); 3.8-4.0 (m, 1 H); 2.8 (s, 3 H); 2.3 (s, 6 H); 1.7-1.9 (m, 2 H); 1.5-1.7 (m, 4 H); 1.2-1.4 (m, 4 H).

ESI-MS: M⁺=502.

The compounds listed in Table 1 can be prepared analogously to the synthesis examples mentioned or as described in the general section.

| | | | | | | | | | | I |
|---|---|---|---|---|---|---|---|---|---|---|
| 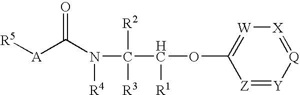 | | | | | | | | | | |
| No. | R¹ | R² | R³ | R⁴ | R⁵A | W | X | Q | Y | Z |
| I-1 | COOMe | 2-Cl—Ph | 2-Cl—Ph | H | MeO | N | C—Me | CH | C—Me | N |
| I-2 | COOH | Ph | Ph | H | EtO | N | C—Me | CH | C—Me | N |
| I-3 | COOH | Ph | Ph | H | EtO | N | N | C—OMe | C—Me | CH |
| I-4 | COOH | Ph | Ph | H | n-PrO | N | C—Me | N | C—Me | CH |
| I-5 | COOH | Ph | Ph | H | 4-MeO—PhO | N | C—Et | C—F | C—OMe | N |
| I-6 | COOH | Ph | 3-Br—Ph | H | 4-Me—PhO | N | C—Et | N | C—Et | N |
| I-7 | COOH | 3-EtO—Ph | 3-EtO—Ph | H | PhCH₂—O | N | C—OMe | CH | C—Me | N |
| I-8 | COOH | Ph | Ph | H | 4-MeO—PhCH₂—O | N | C—OMe | CH | C—OMe | N |
| I-9 | COOH | Ph | Ph | H | 4-NMe₂—PhCH₂—O | N | C—OMe | CH | C—OMe | N |
| I-10 | COOH | 3-Cl—Ph | 3-Cl—Ph | H | 4-Me—Ph—CH₂CH₂—O | N | C—Me | C—CH₂—CH₂—O—C | | N |
| I-11 | COOH | Ph | Ph | H | Me₂N | N | C—OMe | CH | C—OMe | N |
| I-12 | COOH | Ph | Ph | H | Me₂N | N | C—Me | C—CH₂—CH₂—CH₂—C | | N |
| I-13 | COOH | Ph | Ph | H | Et₂N | N | C—Me | CH | C—Me | N |
| I-14 | COOH | Ph | Ph | H | c-Pentyl-NMe | N | C—Me | CH | C—Me | N |
| I-15 | COOH | Ph | Ph | H | c-Hexyl-NMe | N | C—OMe | N | C—Me | CH |
| I-16 | COOH | Ph | Ph | H | PhNMe | CH | C—OMe | N | CH | CH |
| I-17 | COOH | Ph | Ph | H | 4-MeO—PhNMe | N | C—Me | CH | C—Me | N |
| I-18 | COOH | 4-NO₂—Ph | 4-NO₂—Ph | H | 4-Et—PhNMe | N | N | C—OMe | CH | CH |
| I-19 | COOH | Ph | Ph | H | 4-Cl—Ph—NMe | N | C—Me | CH | C—Me | N |
| I-20 | COOH | Ph | Ph | H | 3,4-DiMeO—Ph—NMe | N | C—Me | CH | C—Me | N |
| I-21 | COOH | Ph | Ph | H | PhCH₂—NMe | N | C—OMe | N | C—Et | CH |
| I-22 | COOH | Ph | Ph | H | 4-Me—PhCH₂—NMe | N | C—Et | N | C—Et | N |
| I-23 | COOH | Ph | Ph | H | 4-MeO—PhCH₂—NMe | N | C—OMe | CH | C—OMe | N |
| I-24 | COOH | Ph | Ph | H | 4-F—PhCH₂—NMe | N | C—OMe | CH | C—OMe | N |
| I-25 | COOH | Ph | Ph | H | 3,4-DiMeO—PhCH₂—NMe | N | C—Me | CH | C—Me | N |
| I-26 | COOH | Ph | Ph | H | Pyrrolidine | N | C—Me | CH | C—Me | N |
| I-27 | COOH | 4-Cl—Ph | 4-Cl—Ph | H | Piperidine | N | C—Me | CH | C—Me | N |
| I-28 | COOH | 4-Me—Ph | 4-Me—Ph | H | Piperidine | N | C—OMe | CH | C—OMe | N |
| I-29 | COOEt | Ph | Ph | H | N-Methylpiperazine | N | C—Et | N | C—Et | N |
| I-30 | COOH | Ph | Ph | H | MeO | N | C—Et | CH | C—Et | N |
| I-31 | Tetrazole | Ph | Ph | H | EtO | N | C—Me | CH | C—OMe | N |
| I-32 | CONHSO₂Et | Ph | Ph | H | n-Pro | N | C—Me | CH | C—Me | N |
| I-33 | COOH | Ph | Ph | H | PhO | N | CH | CH | C—OMe | N |
| I-34 | COOH | Ph | Ph | Et | 3,4-DiMe—PhO | N | C—Me | CH | C—Me | N |
| I-35 | COOMe | Ph | Ph | H | PhCH₂—O | N | C—Et | N | C—Et | N |
| I-36 | COOH | Ph | Ph | H | PhCH₂—O | N | C—Me | CH | C-(4-Me—Ph) | N |
| I-37 | COOH | Ph | Ph | H | 3,4-DiMeO—PhCH₂—O | N | C—Et | N | C—Et | N |
| I-38 | COOH | Ph | Ph | H | 4-Me—PhCH₂—O | N | N | C—OMe | CH | CH |
| I-39 | CONHSO₂(4- | Ph | Ph | H | 3,4- | N | C—Me | CH | C—Me | N |
| | MePh) | | | | DiMeO—Ph—CH₂CH₂—O | | | | | |
| I-40 | COOH | Ph | Ph | H | Me₂N | N | C—OEt | N | C—OEt | CH |
| I-41 | COOH | Ph | Ph | H | Et₂N | N | C—Me | C—CH₂—CH₂—S—C | | N |
| I-42 | COOH | Ph | Ph | H | c-Pentyl-NMe | N | C—OEt | N | C—OEt | N |
| I-43 | COOH | 3-Cl—Ph | 3-Cl—Ph | H | 4-MeO—PhNMe | N | C—OMe | C—CH₂—CH₂—CH₂—C | | N |
| I-44 | COOEt | Ph | Ph | H | 2-Me—PhNMe | N | C—Et | CH | CH | N |
| I-45 | COOH | 4-Me—Ph | 4-Me—Ph | H | 3-F—Ph—NMe | N | C—Me | CH | C—Me | N |
| I-46 | COOH | Ph | Ph | H | 4-NMe₂—Ph—NMe | N | C—Me | CH | C—Me | N |
| I-47 | COOH | Ph | Ph | H | PhCH₂—NMe | N | C—Et | N | C—Et | N |
| I-48 | COOH | Ph | Ph | Me | 4-Et—PhCH₂—NMe | N | C—Me | CH | C—Me | N |
| I-49 | COOCH₂Ph | Ph | Ph | H | 3-EtO—PhCH₂—NMe | N | C—Me | N | C—OMe | N |
| I-50 | COOH | Ph | Ph | H | 4-Br—PhCH₂—NMe | N | C—Me | CH | C—Me | N |
| I-51 | COOH | Ph | Ph | H | 3,5-DiMeO—PhCH₂—NMe | N | C—OMe | CH | C—OMe | N |
| I-52 | COOH | Ph | Ph | H | Piperidine | N | C—Me | CH | C—Me | N |
| I-53 | COOH | Ph | Ph | H | Morpholine | N | C—OMe | CH | C—OMe | N |
| I-54 | CONHSO₂(4- | Ph | Ph | H | MeO | N | C—Me | CH | C—Me | N |
| | iPr-Ph) | | | | | | | | | |
| I-55 | COOH | Ph | Ph | Me | EtO | N | C—OMe | CH | C—OMe | N |
| I-56 | COOH | Ph | Ph | H | sec-BuO | N | C—OMe | CH | C—OMe | N |
| I-57 | COOCH₂Ph | Ph | Ph | H | 3,4-DiMe—PhO | N | C—Me | CH | C—Me | N |
| I-58 | Tetrazole | Ph | Ph | H | PhCH₂—O | N | C—OMe | CH | C—Me | N |
| I-59 | COOH | Ph | Ph | H | 3-nPrO—PhCH₂—O | N | C—Me | CH | C—Me | N |
| I-60 | COOEt | Ph | Ph | H | PhCH₂NH | N | C—Me | CH | C—Me | N |
| I-61 | COOH | Ph | Ph | H | Me₂N | N | C—OMe | N | C—OMe | N |
| I-62 | COOMe | Ph | Ph | H | Et₂N | N | C—OMe | CH | C—Et | N |
| I-63 | COOH | Ph | Ph | H | t-BuNMe | N | C—OMe | CH | C—OMe | N |
| I-64 | COOH | 4-NO₂—Ph | 4-NO₂—Ph | Me | c-Hexyl-NMe | N | C—Me | CH | C—Me | N |
| I-65 | COOH | Ph | Ph | H | PhNMe | N | C—Me | CH | C—COOH | N |
| I-66 | COOH | Ph | Ph | H | 3-MeO—PhNMe | N | C—OMe | N | C—OMe | CH |
| I-67 | COOH | Ph | Ph | H | 4-Me—PhNMe | N | C—Me | CH | C—Me | N |
| I-68 | COOH | 3-MeO—Ph | 3-MeO—Ph | H | 4-Cl—Ph—NMe | CH | CH | N | C—OMe | CH |
| I-69 | COOH | Ph | Ph | H | 3,5-DiCl—Ph—NMe | N | C—OMe | C—CH₂—CH₂—CH₂—C | | N |
| I-70 | COOH | Ph | Ph | H | PhCH₂—NMe | N | C—Me | CH | C—Me | CH |
| I-71 | COOH | 3-OH—Ph | 3-OH—Ph | H | 4-MeO—PhCH₂—NMe | N | C—OMe | CH | C—OMe | N |
| I-72 | COOH | c-Hexyl | c-Hexyl | H | 4-Cl—PhCH₂—NMe | N | C—OMe | N | C—Me | CH |
| I-73 | COOH | Ph | 4-Cl—Ph | H | 3,4-DiMeO—PhCH₂—NMe | N | C—OMe | CH | C—Et | N |
| I-74 | COOH | 4-NO₂—Ph | 4-NO₂—Ph | H | Piperidine | N | C—Me | CH | C—Me | N |
| I-75 | COOH | Ph | Ph | H | Morpholine | N | C—Me | CH | C—Me | N |
| I-76 | COOH | Ph | Ph | H | MeO | N | C—OMe | N | C—OMe | N |
| I-77 | COOH | Ph | 4-MeO—Ph | H | MeO | N | C—Me | CH | C—Me | N |
| I-78 | COOH | Ph | Ph | H | n-PrO | N | C—Me | CH | C—OMe | N |
| I-79 | COOH | 3-MeO—Ph | 3-MeO—Ph | H | PhO | N | C—Me | N | C—Me | N |
| I-80 | COOH | Ph | Ph | H | 3-Me—PhO | N | C—OMe | N | C—OMe | CH |
| I-81 | COOH | Ph | Ph | H | 4-NO₂—PhO | N | C—OMe | CH | C—OMe | N |
| I-82 | COOH | 4-F—Ph | 4-F—Ph | Et | 4-MeO—PhCH₂—O | N | C—Me | CH | C—OMe | N |
| I-83 | COOH | Ph | Ph | H | 3-Br—PhCH₂—O | N | C—Et | CH | C—Et | N |
| I-84 | COOH | c-Hexyl | c-Hexyl | H | 3,4- | N | C—OMe | CH | C—Me | N |
| | | | | | DiMeO—Ph—CH₂CH₂—O | | | | | |
| I-85 | COOMe | Ph | Ph | H | Me₂N | N | C—Et | CH | C—Et | N |
| I-86 | COOH | Ph | Ph | H | Et₂N | N | C—OMe | CH | C—OMe | N |
| I-87 | COOH | 4-F—Ph | 4-F—Ph | H | c-Pentyl-NMe | N | C—Me | CH | C—Et | N |
| I-88 | CONHSO₂Me | Ph | 3-nPr—Ph | H | c-Hexyl-NMe | N | C—Me | CH | C—Me | N |
| I-89 | COOH | c-Hexyl | c-Hexyl | H | PhNMe | N | C—OMe | C—Me | CH | N |
| I-90 | COOH | 3-Me—Ph | 3-Me—Ph | H | 3-Me—PhNMe | N | C—Me | CH | C—Me | N |
| I-91 | COOH | Ph | Ph | Me | 4-NO₂—Ph—NMe | N | C—OMe | N | C—OMe | CH |
| I-92 | COOH | Ph | Ph | H | 4-NO₂—PhCH₂—NMe | N | C—Me | CH | C—Me | N |
| I-93 | COOt-Bu | Ph | Ph | Me | Pyrrolidine | N | C—Me | CH | C—Me | N |
| I-94 | COOH | Ph | Ph | H | 4-Methylpiperidine | N | C—Me | CH | C—Me | N |
| I-95 | COOH | c-Hexyl | c-Hexyl | H | MeO | N | C—OMe | CH | C—OMe | N |
| I-96 | CONHSO₂Me | 4-Et—Ph | 4-Et—Ph | H | EtO | N | C—OMe | CH | C—OMe | N |
| I-97 | COOH | Ph | Ph | H | n-PrO | N | C—OMe | CH | C—OMe | N |
| I-98 | CONHSO₂Ph | Ph | Ph | H | 4-NO₂—PhO | N | C—Me | CH | C—Me | N |
| I-99 | CONHSO₂iPr | Ph | Ph | H | 4-MeO—PhCH₂—O | N | C—Me | C—CH═CH—O—C | | N |
| I-100 | COOH | Ph | Ph | H | 4-Cl—Ph—CH₂CH₂—O | N | C—Me | CH | C—Me | N |
| I-101 | COOH | Ph | Ph | H | Me₂N | N | CH | C—OMe | CH | CH |
| I-102 | COOH | c-Hexyl | Ph | H | Et₂N | N | C—Me | CH | C—Me | N |
| I-103 | COOH | Ph | Ph | H | c-Hexyl-NMe | N | C—OMe | CH | C-(4-NMe₂—Ph) | N |
| I-104 | COOH | Ph | Ph | H | PhNMe | N | C—NMe₂ | N | C—NMe₂ | N |
| I-105 | COOH | Ph | Ph | H | PhNMe | N | C—Me | CH | C—Me | N |
| I-106 | COOMe | Ph | Ph | H | 2-PrO—PhNMe | N | C—OMe | CH | C—Me | N |
| I-107 | COOH | 3-EtO—Ph | 3-EtO—Ph | Me | Et₂N | N | C—Me | CH | C—Me | N |
| I-108 | COOH | Ph | Ph | H | 4-NO₂—Ph—CH₂CH₂—O | N | C—Me | CH | C—OMe | N |
| I-109 | COOH | Ph | c-Hexyl | H | 4-MeO—PhCH₂—O | N | C—Me | CH | C—Me | N |
| I-110 | COOH | Ph | Ph | H | 4-MeO—PhO | N | C—OMe | C—CH₂—CH₂—O—C | | N |
| I-111 | COOH | Ph | Ph | H | MeO | N | C—OMe | N | C—Me | CH |
| I-112 | COOH | Ph | Ph | H | 4-MeO—PhCH₂—O | N | C—SMe | CH | C—SMe | N |
| I-113 | COOH | Ph | Ph | H | 3,4-DiCl—PhCH₂—O | N | C—OMe | CH | C—OMe | N |
| I-114 | COOH | Ph | Ph | H | Et₂N | N | C—Me | CH | C—Me | N |
| I-115 | CONHSO₂Me | Ph | Ph | H | PhNMe | N | C—Me | CH | C—Me | N |
| I-116 | COOH | Ph | Ph | H | 4-Me—PhNMe | N | C—OMe | N | C—OMe | N |
| I-117 | COOH | Ph | Ph | H | 4-F—Ph—NMe | N | C—OMe | C—CH₂—CH₂—O—C | | N |
| I-118 | COOH | c-Hexyl | c-Hexyl | H | 3,5-DiMeO—Ph—NMe | N | C—OMe | CH | C—OMe | N |
| I-119 | COOH | 4-F—Ph | 4-F—Ph | H | 4-Me—PhCH₂—NMe | N | C—OMe | CH | C—OMe | N |
| I-120 | COOH | Ph | Ph | H | 4-CN—Ph—NMe | N | C—OMe | CH | C—OMe | N |
| I-121 | COOH | Ph | Ph | H | 4-MeO—PhNMe | N | C—OMe | CH | C—OMe | N |
| I-122 | COOPr | Ph | Ph | H | c-Pentyl-NMe | N | N | CH | C—OMe | CH |
| I-123 | COOEt | Ph | Ph | H | 4-EtO—PhCH₂—O | N | OMe | N | OMe | N |
| I-124 | COOH | 3-Et—Ph | 3-Et—Ph | H | 3,4-DiMeO—PhCH₂—O | N | C—OMe | CH | C—OMe | N |
| I-125 | COOH | 4-Cl—Ph | 4-Cl—Ph | H | PhCH₂—O | N | C—OMe | CH | C—OMe | N |
| I-126 | COOH | Ph | Ph | H | 3-Me—PhO | N | C—OMe | CH | C—Me | N |
| I-127 | COOH | Ph | Ph | H | PhO | N | C—Et | N | C—Et | CH |
| I-128 | COOH | 4-Me—Ph | 4-Me—Ph | H | 3,4-DiMe—PhO | N | C—OMe | CH | C—OMe | N |
| I-129 | COOH | Ph | Ph | H | PhCH₂—O | N | C—OMe | N | C—OMe | CH |
| I-130 | COOt—Bu | Ph | Ph | H | 3-MeO—Ph—CH₂CH₂—O | N | C—OMe | N | C—OMe | CH |
| I-131 | COOH | Ph | Ph | H | c-Heptyl-NMe | N | C—OMe | CH | C—OMe | N |
| I-132 | COOH | 3,4- | 3,4- | H | PhNMe | N | C—Me | CH | C—OMe | N |
| | | DiMeO—Ph | DiMeO—Ph | | | | | | | |
| I-133 | COOH | 1-Nphth | 1-Nphth | H | 4-MeO—PhNMe | N | C—OMe | CH | C—OMe | N |
| I-134 | COOH | Ph | Ph | H | PhCH₂—NMe | N | C—Me | CH | C—Me | N |
| I-135 | COOH | 4-CN—Ph | 4-CN—Ph | H | 3,4-DiMe—Ph—NMe | N | C—OMe | CH | C—OMe | N |
| I-136 | CONHSO₂Ph | Ph | Ph | H | 2-Me—PhNMe | N | C—OMe | CH | N | CH |
| I-137 | COOH | Ph | Ph | H | PhNMe | N | C—Et | N | C—OMe | CH |
| I-138 | CONHSO₂Et | Ph | Ph | H | t-BuNMe | N | C—Et | CH | C—Et | N |
| I-139 | COOH | Ph | Ph | H | 2-NO₂—4,5- | N | C—Me | CH | C—Me | N |
| | | | | | DiMeO—PhCH₂—O | | | | | |
| I-140 | COOH | Ph | Ph | H | PhCH₂—O | N | C—OMe | CH | C—OMe | N |
| I-141 | COOH | Ph | Ph | H | 3,4-DiMeO—PhCH₂—O | N | C—Me | CH | C—Me | N |
| I-142 | CONHSO₂(4- | Ph | Ph | H | Me₂N | N | C—Me | CH | C—Me | N |
| | iPr-Ph) | | | | | | | | | |
| I-143 | COOH | Ph | Ph | H | 4-EtO—PhNMe | N | C—Me | CH | C—SMe | N |
| I-144 | COOt-Bu | Ph | 4-Et—Ph | H | 4-Cl—Ph—NMe | N | C—Et | CH | C—Ph | N |
| I-145 | COOH | 4-Et—Ph | 4-Et—Ph | H | 3-MeO—PhNMe | N | C—Me | C—CH₂—CH₂—O—C | | N |
| I-146 | COOH | Ph | Ph | Me | 3-Et—PhNMe | N | C—Me | C—CH₂—CH₂—S—C | | N |
| I-147 | COOH | 4-Et—Ph | 4-Et—Ph | H | Me₂N | N | C—Me | CH | C—Me | N |
| I-148 | COOH | Ph | Ph | H | 3,4-DiMeO—Ph—NMe | N | CH | C—Me | C—Et | N |
| I-149 | COOH | Ph | Ph | H | 3-Cl—PhCH₂—NMe | N | C—Me | CH | C-(4-MeO—Ph) | N |
| I-150 | COOEt | Ph | Ph | H | 2-MeO—PhCH₂—NMe | N | C—OMe | CH | C—OMe | N |
| I-151 | COOH | Ph | Ph | H | 3,4-OCH₂O—PhCH₂—NMe | N | C—Me | CH | C—Me | N |
| I-152 | COOH | 4-MeO—Ph | 4-MeO—Ph | H | MeO | N | C—OMe | CH | C—Me | N |
| I-153 | COOH | Ph | Ph | H | 2-Cl—PhO | N | C—Et | CH | C—Et | N |
| I-154 | COOH | c-Hexyl | c-Hexyl | H | Me₂N | N | C—Me | CH | C—OMe | N |
| I-155 | COOH | Ph | Ph | H | PhNMe | N | C—OMe | CH | C—OMe | N |
| I-156 | COOH | Ph | Ph | H | 4-NO₂—Ph—NMe | N | C—Me | CH | C—Et | N |
| I-157 | COOH | Ph | Ph | H | 3-MeO—PhCH₂—NMe | N | C—Me | CH | C—Me | N |
| I-158 | COOH | Ph | Ph | H | Pyrrolidine | N | C—OMe | CH | C—OMe | N |
| I-159 | COOH | Ph | Ph | H | Pyrrolidine | N | C—Me | CH | C-(4-Cl—Ph) | N |
| I-160 | COOH | Ph | c-Hexyl | H | Piperidine | N | C—OMe | CH | C—OMe | N |
| I-161 | COOH | 2-Nphth | 2-Nphth | Me | Morpholine | N | C—Me | CH | C—Me | N |
| I-162 | COOH | Ph | Ph | H | N-Methylpiperazine | N | C—Me | CH | C—Me | N |
| I-163 | COOH | 3-EtO—Ph | 3-EtO—Ph | H | PhCH₂—NMe | N | C—Me | C—CH₂—CH₂—O—C | | N |
| I-164 | COOH | c-Pentyl | c-Pentyl | H | 4-Me—PhNMe | N | C—OMe | C—F | C—OMe | N |
| I-165 | COOH | Ph | Ph | H | t-BuNMe | N | C—Me | CH | C—Me | N |
| I-166 | CONHSO₂Et | Ph | Ph | H | PhCH₂NH | N | C—Me | C—CH₂—CH₂—O—C | | N |
| I-167 | COOH | Ph | Ph | H | 3-Me—PhCH₂—O | CH | C—OMe | N | C—OMe | CH |
| I-168 | COOH | Ph | Ph | H | 3,4-DiMe—PhO | N | C—OMe | CH | C—OMe | N |
| I-169 | COOH | Ph | 4-Et—Ph | H | PrO | N | C—Me | CH | C-(4-Me—Ph) | N |
| I-170 | COOH | Ph | Ph | H | 4-MeO—PhO | N | C—OMe | CH | C—OMe | N |
| I-171 | COOH | 2-Nphth | Ph | H | EtO | N | C—OMe | CH | C—Me | N |
| I-172 | COOH | Ph | Ph | H | 3,5-DiMeO—Ph—NMe | N | C—Et | N | C—Et | N |
| I-173 | COOH | Ph | Ph | H | 3-F—Ph—NMe | N | C—Me | N | C—Me | N |
| I-174 | COOH | Ph | Ph | H | 4-EtO—PhCH₂—NMe | N | C—OMe | CH | C—OMe | N |
| I-175 | COOH | 4-MeS—Ph | 4-MeS—Ph | H | Pyrrolidine | N | C—Me | CH | C—Me | N |
| I-176 | COOH | Ph | Ph | H | Piperidine | N | C—OMe | CH | C—OMe | N |
| I-177 | COOCH₂Ph | Ph | Ph | H | Morpholine | N | C—OMe | CH | C—OMe | N |
| I-178 | COOH | Ph | 2-Nphth | H | PhCH₂—NMe | N | C—Me | C—CH₂—CH₂—CH₂—C | | N |
| I-179 | CONHSO₂c-Hexyl | Ph | Ph | H | 4-EtO—PhNMe | N | C—Me | CH | C—OMe | N |
| I-180 | COOH | Ph | Ph | H | Ph—CH₂CH₂—O | N | C—OMe | CH | C—OMe | N |
| I-181 | COOH | Ph | Ph | H | PrO | N | C—OMe | C—CH₂—CH₂—CH₂—C | | N |
| I-182 | COOH | 4-MeO—Ph | 4-MeO—Ph | H | 4-CN—PhCH₂—NMe | N | C—Me | C—CH₂—CH₂—O—C | | N |
| I-183 | COOH | Ph | Ph | H | 3,4-DiMe—PhCH₂—NMe | N | C—OMe | CH | C—Me | N |
| I-184 | CONHSO₂Ph | Ph | Ph | H | 3,4-DiCl—PhCH₂—NMe | N | C—Me | CH | C—Me | N |
| I-185 | COOH | 3-Br—Ph | Ph | H | N-Methylpiperazine | N | C—Me | CH | C—Me | N |
| I-186 | COOH | 3-Et—Ph | 3-Et—Ph | H | Pyrrolidine | N | CH | C—F | C—Et | N |
| I-187 | COOH | Ph | Ph | H | 4-Me—PhCH₂—NMe | N | C—Me | CH | C—Me | N |
| I-188 | COOH | Ph | Ph | H | 2-F—Ph—NMe | N | C—Et | CH | C—Et | N |
| I-189 | COOH | Ph | Ph | H | Me₂N | N | C—Me | CH | C-(4-MeO—Ph) | N |
| I-190 | COOMe | Ph | Ph | H | 3,4-DiMeO—PhCH₂—O | N | C—OMe | CH | C—OMe | N |
| I-191 | COOEt | Ph | Ph | Me | 4-MeO—PhNMe | N | C—Me | CH | C—Ph | N |
| I-192 | COOH | Ph | Ph | H | 4-Me—PhNMe | N | C—OMe | C—F | C—OMe | N |
| I-193 | Tetrazole | Ph | Ph | H | PhCH₂—NMe | N | C—Me | CH | C—Me | N |
| I-194 | COOH | Ph | Ph | H | 4-MeO—PhCH₂—NMe | N | C—Me | CH | C—Me | N |
| I-195 | COOCH₂Ph | Ph | Ph | H | 3,4-DiMeO—PhCH₂—NMe | N | C—OMe | N | C—OMe | CH |
| I-196 | CONHSO₂Et | 3-F—Ph | 3-F—Ph | H | Morpholine | N | C—Et | CH | C—Et | N |
| I-197 | COOH | Ph | Ph | H | Pyrrolidine | N | C—OMe | CH | C—OMe | N |
| I-198 | COOH | Ph | Ph | H | 2,4-DiMeO—Ph—NMe | N | C—OMe | C—F | C—OMe | N |
| I-199 | COOH | 4-Cl—Ph | 4-Cl—Ph | H | PhCH₂—NMe | N | C—Me | CH | C—OMe | N |
| I-200 | COOH | Ph | Ph | H | c-Pentyl-NMe | N | C—OMe | CH | CH | N |
| I-201 | COOH | 4-Br—Ph | Ph | H | 4-MeO—PhNMe | N | C—Et | CH | C—Et | N |
| I-202 | COOH | Ph | Ph | H | c-Hexyl-NMe | N | C—Me | CH | C—Me | N |
| I-203 | CONHSO₂c-Hexyl | Ph | 4-NO₂—Ph | H | PhO | N | C—Me | CH | C—OMe | N |
| I-204 | CONHSO₂(4-MePh) | Ph | Ph | H | PhCH₂—O | N | C—Me | CH | C—Me | N |
| I-205 | COOMe | Ph | Ph | H | H₂N | N | C—OMe | CH | C—OMe | N |
| I-206 | COOCH₂Ph | Ph | Ph | H | Et₂N | N | C—Et | N | C—Et | N |
| I-207 | COOH | 3-F—Ph | Ph | H | c-Pentyl-NMe | N | C—OMe | CH | C—OMe | N |
| I-208 | COOH | Ph | Ph | H | EtO | N | C—Et | N | C—Et | N |
| I-209 | COOEt | Ph | Ph | H | n-PrO | N | C—OMe | CH | C—OMe | N |
| I-210 | COOH | Ph | Ph | H | MeO | N | C—Me | CH | C—Me | N |
| I-211 | COOH | Ph | Ph | H | 4-Me—PhCH₂—O | N | C—Me | CH | C—Me | N |
| I-212 | COOH | 3-Br—Ph | 3-Br—Ph | H | t-BuNMe | N | C—Me | CH | C—OMe | N |
| I-213 | COOH | Ph | Ph | H | 3,4-DiCl—Ph—NMe | N | C—Me | CH | C—Me | N |
| I-214 | COOH | Ph | Ph | H | 4-MeO—PhCH₂—NMe | N | C—Me | CH | C—SEt | N |
| I-215 | CONHSO₂Me | Ph | Ph | H | 4-Cl—PhCH₂—NMe | N | C—Me | CH | C—Me | N |
| I-216 | COOEt | Ph | Ph | H | 4-F—Ph—NMe | N | C—OMe | CH | C—OMe | N |
| I-217 | COOH | Ph | Ph | H | 3,4-OCH₂O—PhCH₂—NMe | N | CH | CH | C—Et | N |
| I-218 | COOH | Ph | Ph | H | Pyrrolidine | N | C—OMe | C—CH₂—CH₂—CH₂—C | | N |
| I-219 | COOH | Ph | Ph | H | Morpholine | CH | C—Me | N | C—OMe | CH |
| I-220 | COOH | Ph | Ph | H | 3-Cl—Ph—NMe | N | CH | C—Me | C—OMe | N |
| I-221 | COOH | 3,4- | Ph | H | Me₂N | N | C—OMe | CH | C—OMe | N |
| | | DiMeO—Ph | | | | | | | | |
| I-222 | COOH | Ph | Ph | H | 3,4- | N | C—OMe | CH | C—OMe | N |
| | | | | | DiMeO—Ph—CH₂CH₂—O | | | | | |
| I-223 | COOCH₂Ph | Ph | Ph | H | 3-Me-4-MeO—Ph—NMe | N | C—Me | CH | C—Me | N |
| I-224 | COOH | Ph | Ph | Me | 4-NMe₂—PhCH₂—NMe | N | C—OMe | CH | C—OMe | N |
| I-225 | COOH | Ph | 2-Nphth | H | Pyrrolidine | N | C—OMe | CH | C—Me | N |
| I-226 | COOH | Ph | Ph | H | 4-Cl—PhCH₂—NMe | N | C—OMe | N | C—OMe | N |
| I-227 | COOH | Ph | Ph | H | Me₂N | N | C—Me | CH | C—Me | N |
| I-228 | COOH | Ph | Ph | H | 4-nPrO—PhCH₂—NMe | N | C—OMe | C—CH₂—CH₂—CH₂—C | | N |
| I-229 | COOH | Ph | Ph | H | n-BuO | N | C—Et | CH | C—Et | N |
| I-230 | COOH | Ph | Ph | H | MeO | N | C—OMe | CH | C—OMe | N |
| I-231 | COOEt | Ph | Ph | H | PhO | N | C—Me | CH | C—OMe | N |
| I-232 | COOH | Ph | Ph | H | 4-Me—PhO | N | C—Et | CH | C—OMe | N |
| I-233 | COOH | Ph | Ph | H | PhCH₂—O | N | C—Me | C—CH₂—CH₂—O—C | | N |
| I-234 | COOH | Ph | Ph | H | 4-MeO—PhCH₂—O | CH | C—Me | N | C—Me | CH |
| I-235 | Tetrazole | Ph | Ph | H | Me₂N | N | C—OMe | CH | C—OMe | N |
| I-236 | COOH | Ph | Ph | H | t-BuNH | N | C—Me | CH | C—Me | N |
| I-237 | COOEt | 4-OH—Ph | 4-OH—Ph | H | c-Hexyl-NH | N | C—OMe | CH | C—Me | N |
| I-238 | Tetrazole | Ph | Ph | H | 4-MeO—PhNMe | N | C—Me | CH | C—Me | N |
| I-239 | COOH | Ph | Ph | H | 4-OH—Ph—NMe | N | C—Et | C—CH₂—CH₂—O—C | | N |
| I-240 | COOH | Ph | Ph | H | 4-MeO—PhCH₂—NMe | N | N | C—OMe | CH | N |
| I-241 | COOH | Ph | Ph | H | Piperidine | N | C—Me | CH | C-(4-MeO—Ph) | N |
| I-242 | COOH | Ph | Ph | H | N-Methylpiperazine | N | C—OMe | CH | C—OMe | N |
| I-243 | COOH | Ph | Ph | H | c-Hexyl-NMe | N | C—OMe | C—CH₂—CH₂—O—C | | N |
| I-244 | COOH | Ph | Ph | H | 3-Cl—PhO | N | C—OMe | CH | C—OMe | N |
| I-245 | COOH | Ph | Ph | H | PhCH₂—O | N | C—Me | CH | C—Me | N |
| I-246 | COOH | 3-CN—Ph | 3-CN—Ph | H | Me₂N | N | C—OMe | CH | C—Me | N |
| I-247 | COOH | Ph | Ph | H | Et₂N | CH | C—OMe | N | C—Me | CH |
| I-248 | COOH | 3-F—Ph | 3-F—Ph | H | n-PrO | N | C—Et | N | C—Et | N |
| I-249 | COOH | Ph | Ph | H | Piperidine | N | C—Me | CH | C—COOH | N |
| I-250 | COOMe | Ph | Ph | H | 4-F—PhCH₂—NMe | CH | C—Me | N | CH | CH |
| I-251 | COOc-Hexyl | Ph | Ph | H | PhCH₂—NMe | N | C—Me | CH | C—Me | N |
| I-252 | COOH | 3-Br—Ph | 3-Br—Ph | H | 3-MeO—PhCH₂—NMe | N | C—Me | C—CH₂—CH₂—O—C | | N |
| I-253 | COOH | Ph | Ph | Me | Ph—CH₂CH₂—O | N | C—Me | CH | C—OMe | N |
| I-254 | COOH | Ph | Ph | H | EtO | N | C—OMe | CH | C—OMe | N |
| I-255 | COOH | c-Hexyl | c-Hexyl | H | 4-MeO—PhO | N | C—Me | CH | C—Me | N |
| I-256 | COOH | Ph | Ph | H | MeO | N | C—Me | C—CH₂—CH₂—O—C | | N |
| I-257 | COOH | 4-NO₂—Ph | 4-NO₂—Ph | H | PhCH₂—O | N | C—OMe | CH | C—OMe | N |
| I-258 | COOH | Ph | Ph | H | 4-NO₂—PhCH₂—O | N | C—OMe | CH | C—Me | N |
| I-259 | COOH | Ph | Ph | H | 4-MeO—PhNMe | N | C—Et | N | C—Et | N |
| I-260 | COOH | Ph | Ph | H | PhCH₂—NMe | N | C—OMe | CH | C—OMe | N |
| I-261 | COOH | Ph | Ph | H | 4-Carboxy-PhCH₂—NMe | N | C—Me | CH | C—Me | N |
| I-262 | COOH | Ph | Ph | H | 2-Methylpiperidine | N | C—OMe | C—CH₂—CH₂—O—C | | N |
| I-263 | COOH | Ph | Ph | H | 2-Methylpyrrolidine | N | C—OMe | CH | C—OMe | N |
| I-264 | COOH | Ph | Ph | H | Morpholine | N | C—Et | N | C—Et | N |
| I-265 | CONHSO₂(4-iPr- | Ph | Ph | H | 3-Br—Ph—NMe | N | C—OMe | CH | C—Me | N |
| | Ph) | | | | | | | | | |
| I-266 | COOH | Ph | Ph | H | 2-NO₂—4,5- | N | C—OMe | CH | C—OMe | N |
| | | | | | DiMeO—PhCH₂—O | | | | | |
| I-267 | COOH | Ph | Ph | H | 3,4- | N | C—NMe₂ | N | C—NMe₂ | N |
| | | | | | DiMeO—Ph—CH₂CH₂—O | | | | | |
| I-268 | COOH | Ph | Ph | H | 3-Me—PhCH₂—NMe | N | C—OMe | N | C—OMe | CH |
| I-269 | COOEt | Ph | 3-Cl—Ph | H | EtO | N | C—Me | C—CH₂—CH₂—O—C | | N |
| I-270 | COOH | Ph | Ph | H | PhCH₂—O | N | C—Me | CH | C—COOH | N |
| I-271 | COOH | Ph | Ph | H | Piperidine | N | C—OMe | N | C—OMe | CH |
| I-272 | COOH | Ph | Ph | H | PhO | N | C—OMe | CH | C—OMe | N |
| I-273 | COOH | Ph | Ph | H | PrO | N | C—OMe | CH | C—OMe | N |
| I-274 | COOMe | Ph | 4-Me—Ph | H | 4-Br—PhCH₂—O | N | C—OMe | N | C—OMe | CH |
| I-275 | COOH | Ph | Ph | H | 3-Et—PhCH₂—NMe | N | C—Et | C—F | C—Et | N |
| I-276 | COOEt | Ph | Ph | Me | Piperidine | N | C—Me | CH | C—Me | N |
| I-277 | COOH | 2-Nphth | 2-Nphth | H | EtO | N | C—Me | CH | C—Me | N |
| I-278 | COOH | Ph | Ph | H | MeO | N | C—Me | CH | C-(4-MeO—Ph) | N |
| I-279 | COOH | Ph | Ph | H | EtO | N | C—Me | C—OMe | CH | CH |
| I-280 | COOH | 2-Nphth | 2-Nphth | H | PhNMe | N | C—OMe | C—Br | C—OMe | N |
| I-281 | COOH | Ph | Ph | H | 3-Me-4-MeO—PhCH₂—NMe | N | C—OMe | CH | C—Me | N |
| I-282 | CO-2- | Ph | Ph | Me | EtO | N | C—Me | CH | C—Me | N |
| | Imidazolyl | | | | | | | | | |
| I-283 | COOH | Ph | Ph | H | 4-MeO—PhCH₂NMe | N | C—C═CH | CH | C—OMe | N |
| I-284 | COOH | Ph | Ph | H | 3,4-DiOMe—PhO | N | C—CF₃ | CH | C—Me | N |
| I-285 | COOCH₂CH₂SO₂Et | 3-EtO—Ph | 3-EtO—Ph | H | Me₂N | N | C—OMe | CH | C—OMe | N |
| I-286 | COOH | Ph | Ph | H | n-PrO | N | C—CH₂OH | CH | C—Et | N |
| I-287 | COOH | Ph | Ph | H | PhCH₂NMe | N | C—OMe | C—OMe | C—OMe | N |
| I-288 | COOH | Ph | Ph | H | 4-MeO—PhCH₂NMe | N | C—Me | CH | C-OAllyl | N |
| I-289 | COOH | 4-Br—Ph | 4-Br—Ph | H | 3,4-Di-MeO—PhCH₂O | N | C—Cl | CH | C—Me | N |
| I-290 | COOEt | Ph | Ph | Et | 4-BrPhCH₂NMe | N | C—Et | N | C—OCF₃ | N |
| I-291 | COOH | Ph | Ph | H | Pyrrolidine | N | C—Me | C—Me | C—OMe | N |
| I-292 | COOH | Ph | Ph | H | Cyclohexyl-N—Me | N | ″ | CH | C—O—CH₂CH₂OH | N |
| I-293 | COOH | Ph | Ph | H | Et₂N | N | C—OMe | CH | C—O—CH₂CH₂OH | N |

According to the binding test described in the general section, receptor binding data were measured for the compounds listed below. The results are shown in Table 2.

TABLE 2
Receptor binding data (Kᵢ values)

| Compound | ET_{A} [nM] | ET_{B} [nM] |
|---|---|---|
| I-11 | 397 | >10000 |
| I-23 | 147 | 6620 |
| I-26 | 53 | 3850 |
| I-52 | 73 | 4220 |
| I-75 | 117 | 3450 |
| I-86 | 253 | >10000 |
| I-121 | 310 | 10000 |
| I-140 | 97 | 2340 |
| I-155 | 265 | 5770 |
| I-168 | 105 | 730 |
| I-170 | 120 | 1930 |
| I-194 | 14 | 2330 |
| I-202 | 19 | 195 |
| I-230 | 89 | >10000 |
| I-260 | 235 | 2440 |
| I-266 | 13 | 1090 |

we claim:

1. A carbamate or urea derivative of the formula I 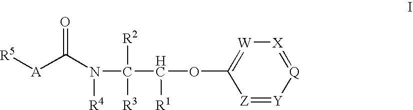
where the substituents have the following meanings:
R¹ is tetrazole or a group 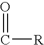
in which R is:
a) a radical OR⁶, in which R⁶ is:
hydrogen, the cation of an alkali metal, the cation of an alkaline earth metal, a physiologically tolerable organic ammonium ion such as tertiary C₁-C₄-alkylammonium or the ammonium ion;
C₃-C₈-cycloalkyl, C₁-C₈-alkyl, CH₂-phenyl, each of which can optionally be substituted,
a C₂-C₆-alkenyl group or a C₃-C₆-alkynyl group, where these groups for their part can carry one to five halogen atoms;
R⁶ can furthermore be an optionally substituted phenyl radical;
b) pyrrolyl, pyrazolyl, imidazolyl and triazolyl, which can carry one or two halogen atoms, or one or two C₁-C₄-alkyl groups or one or two C₁-C₄-alkoxy groups;
c) a group 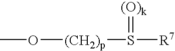
where
k=0, 1 or 2;
p=1, 2, 3 or 4;
R⁷ is C₁-C₄-alkyl, C₃-C₈-cycloalkyl, C₂-C₆-alkenyl, C₂-C₆-alkynyl or phenyl, which can optionally be mono- or polysubstituted;
d) a radical 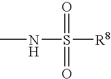
R⁸ is C₁-C₄-alkyl, C₂-C₆-alkenyl, C₂-C₆-alkynyl, C₃-C₈-cycloalkyl, where these radicals can carry a C₁-C₄-alkoxy radical, C₁-C₄-alkylthio radical and/or an optionally substituted phenyl radical;
phenyl, which is optionally substituted;
R² and R³ (which can be identical or different) are:
phenyl or naphthyl, each of which can be substituted by one or more of the following radicals: halogen, nitro, cyano, hydroxyl, mercapto, C₁-C₄-alkyl, C₂-C₄-alkenyl, C₂-C₄-alkynyl, C₁-C₄-haloalkyl, C₁-C₄-alkoxy, phenoxy, C₁-C₄-haloalkoxy, C₁-C₄-alkylthio, amino, NH(C₁-C₄-alkyl), N(C₁-C₄-alkyl)₂ or phenyl, which can be mono- or polysubstituted by halogen, nitro, cyano, C₁-C₄-alkyl, C₁-C₄-haloalkyl, C₁-C₄-alkoxy, C₁-C₄-haloalkoxy or C₁-C₄-alkylthio;
phenyl or naphthyl, which are bonded to one another in the ortho position via a direct bond, a methylene, ethylene or ethenylene group, an oxygen or sulfur atom or an SO₂—, NH— or N-alkyl group;
C₅-C₆-cycloalkyl;
R⁴ is hydrogen, C₁-C₄-alkyl;
R⁵ is C₁-C₈-alkyl which is optionally substituted;
C₃-C₈-cycloalkyl which is optionally substituted;
phenyl or naphthyl, each of which can carry one to three of the following substituents: halogen, cyano, C₁-C₄-alkoxy, C₁-C₄-alkyl, C₁-C₄-alkylthio, NH(C₁-C₄-alkyl), N(C₁-C₄-alkyl)₂, amino or carboxyl; or R⁵ forms a three- to seven-membered ring with NR⁹ as indicated under R⁹;
A is oxygen or NR⁹ where
R⁹ is hydrogen, C₁-C₄-alkyl;
or NR⁹ forms a three- to seven-membered saturated ring, which can be substituted by C₁-C₄-alkyl and in which up to two of the methylene groups can be replaced by oxygen, sulfur, NH or N(C₁-C₄-alkyl), with R⁵ and an appropriate number of methylene groups;
w and Z (which can be identical or different) are:
nitrogen or methine; with the proviso that if W and Z=methine, then Q=nitrogen;
X is nitrogen or CR¹⁰;
Y is nitrogen or CR¹¹;
Q is nitrogen or CR¹²; with the proviso that if Q=nitrogen, then X=CR¹⁰ and Y=CR¹¹; with the proviso that at most three of the ring members designated by W, X, Q, Y and Z are nitrogen;
R¹⁰ and R¹¹ (which can be identical or different) are:
hydrogen, halogen, C₁-C₄-haloalkoxy, C₃-C₆-alkenyloxy, C₃-C₆-alkynyloxy, C₁-C₄-alkylthio, C₁-C₄-alkylcarbonyl, C₁-C₄-alkoxycarbonyl, hydroxyl, NH₂, NH(C₁-C₄-alkyl), N(C₁-C₄-alkyl)₂, carboxyl;
C₁-C₄-alkyl, C₂-C₄-alkenyl, C₂-C₄-alkynyl, where these radicals can be mono- or polysubstituted by halogen, hydroxyl, mercapto, carboxyl, cyano, phenyl, C₁-C₄-alkoxy;
phenyl or phenoxy, which is optionally mono- or disubstituted; C1-C4-alkoxy, optionally substituted; or CR¹⁰ or CR¹¹ is linked with CR¹² as indicated under R¹² to give a 5- or 6-membered ring;
R¹² is hydrogen, halogen, C₁-C₄-alkoxy, C₁-C₄-haloalkoxy, C₁-C₄-alkylthio, C₁-C₄-alkylcarbonyl, C₁-C₄-alkoxycarbonyl, NH(C₁-C₄-alkyl), N(C₁-C₄-alkyl)₂, hydroxyl, carboxyl, cyano, amino, mercapto;
C₁-C₄-alkyl, C₂-C₄-alkenyl, C₂-C₄-alkynyl, where these radicals can be mono- or polysubstituted by: halogen, hydroxy, mercapto, carboxyl, cyano, amino, C₁-C₄-alkoxy;
or CR¹² forms a 5- or 6-membered alkylene or alkenylene ring, which can be substituted by one or two C₁-₄-alkyl groups, and in which one or more methylene groups in each case can be replaced by oxygen, sulfur, —NH or —N(C₁-C₄-alkyl), together with CR¹⁰ or CR¹¹.

2. The use of the carbamate and urea derivatives I as claimed in claim 1 for the production of drugs.

3. The use of the compounds I as claimed in claim 2 as endothelin receptor antagonists.

4. The use as claimed in claim 2 for the treatment of illnesses in which raised endothelin levels occur.

5. The use as claimed in claim 2 for the treatment of illnesses in which endothelin contributes to the origin and/or progression.

6. The use as claimed in claim 2 for the treatment of chronic cardiac insufficiency, restenosis, high blood pressure, pulmonary hypertension, acute/chronic kidney failure, impaired erection, cirrhosis of the liver, cerebral ischemia, benign prostate hyperplasia, acute pancreatitis and prostate cancer.

7. A combination of the carbamate or urea derivative I as claimed in claim 1 and one or more active compounds selected from inhibitors of the renin-angiotensin system, beta-blockers, diuretics, calcium antagonists and VEGF blocking substances for the treatment of high blood pressure.

8. A pharmaceutical preparation for peroral and parenteral administration, comprising, in addition to the customary pharmaceutical excipients, at least one carbamate or urea derivative I as claimed in claim 1.
